SEQUENCE LISTING (18 sequences)

SEQ ID NO: 1 - protein, 8 aa, Artificial Sequence
  Example spacer sequence
GGSGGSGG

SEQ ID NO: 2 - protein, 7 aa, Artificial Sequence
  Example Affinity Tag
NNPMHQN

SEQ ID NO: 3 - DNA, 38 bp, Artificial Sequence
  A-ssDNA, 3′-terminal Biotin
cttgtgtctacttccaatccaatttttttttttttttt

SEQ ID NO: 4 - DNA, 29 bp, Artificial Sequence
  A′-ssDNA, 3′- terminal sulfhydryl linkered
tacttccaatccaattttttttttttttt

SEQ ID NO: 5 - DNA, 30 bp, Artificial Sequence
  B-ssDNA, 3′ terminal sulfhydryl linkered
ttctctacactgtctttttttttttttttt

SEQ ID NO: 6 - DNA, 33 bp, Artificial Sequence
  B′C′-ssDNA
agacagtgtagagaaaatattgataaggatagc

SEQ ID NO: 7 - DNA, 30 bp, Artificial Sequence
  C-ssDNA
ttttttttttttgctatccttatcaatatt

SEQ ID NO: 8 - DNA, 23 bp, Artificial Sequence
  A′′-ssDNA
attggattggaagtagacacaag

SEQ ID NO: 9 - DNA, 15 bp, Artificial Sequence
  C′′-ssDNA
atccttatcaatatt

SEQ ID NO: 10 - DNA, 27 bp, Artificial Sequence
  Linker side
gctatccttatcaatattcgacctatc

SEQ ID NO: 11 - DNA, 57 bp, Artificial Sequence
  Target DNA
agacagtgtgcagaagatggatcggttgctgataggtcgaatttaagtaagtagagc

SEQ ID NO: 12 - DNA, 18 bp, Artificial Sequence
  NP attached
gctatccttatcaatatt

SEQ ID NO: 13 - DNA, 15 bp, Artificial Sequence
  NP-attached
ttctctacactgtct

SEQ ID NO: 14 - DNA, 27 bp, Artificial Sequence
  Sensor H-3end
gctactcttactaatattcgacctcga

SEQ ID NO: 15 - DNA, 27 bp, Artificial Sequence
  Sensor H-3mid
gctactcttactaatattctcactatc

SEQ ID NO: 16 - DNA, 27 bp, Artificial Sequence
  Sensor H-1mid
gctactcttactaatattcgccctatc

SEQ ID NO: 17 - DNA, 27 bp, Artificial Sequence
  Sensor H-5end
gctactcttactaatattcgacagcga

SEQ ID NO: 18 - DNA, 27 bp, Artificial Sequence
  Sensor R-3mid
gctactaggactaatattcgacctact

(19) United States
(12) Patent Application Publication
(10) Pub. No.: US 2009/0258355 A1
(43) Pub. Date: Oct. 15, 2009

(54) Nanoscale Clusters and Methods of Making Same

(75) Inventors: Mathew M. Maye, Binghamton, NY (US); Oleg Gang, South Setauket, NY (US); Dmytro Nykypanchuk, Westbury, NY (US); Daniel van der Lelie, Shoreham, NY (US)

Correspondence Address: BROOKHAVEN SCIENCE ASSOCIATES/;BROOKHAVEN NATIONAL LABORATORY, BLDG. 185 - P.O. BOX 5000, UPTON, NY, 11973, US

(73) Assignee: Brookhaven Science Associates, LLC, Upton, NY (US)

(21) Appl. No.: 12/263,989

(22) Filed: Nov. 3, 2008

Related U.S. Application Data
(60) Provisional application No. 61/044,224, filed on Apr. 11, 2008.

(51) Int. Cl. C12Q 1/68; B82B 3/00

(52) U.S. Cl. 435/6; 977/920; 977/840

(57) ABSTRACT

The present invention is a method of making a nanocluster. The method comprises providing a surface comprising at least one anchoring biomolecule, wherein the surface is in a solution; adding an initial recognition-nano-component to the solution wherein the initial recognition-nano-component comprises i) a nanoparticle and one specifically-bindable-biomolecule, or ii) a nanoparticle and two different types of specifically-bindable-biomolecules, wherein a biomolecule of the initial recognition-nano-component specifically binds to the anchoring biomolecule; and adding a releasing biomolecule to the solution, wherein the releasing biomolecule binds to the anchoring biomolecule with a greater binding strength than the anchoring biomolecule binds to the initial recognition-nano-component, or wherein the releasing biomolecule binds to the initial recognition-nano-component with a greater binding strength than anchoring biomolecule binds to the initial recognition-nano-component, thereby making a nanocluster.

This invention was made with Government support under contract number DE-AC02-98CH10886, awarded by the U.S. Department of Energy. The Government has certain rights in the invention.

### BACKGROUND OF THE INVENTION

Self-assembled nanosystems consisting of inorganic “building blocks” are typically driven by a symmetric functionalization which limits control over assembly morphology and binding properties. Thus, the ability to easily fabricate discrete blocks with anisotropic interactions may aid in the creation of diverse classes of nanoparticulate monomers, clusters, or groupings, which is a current goal of soft nanotechnology.

The use of DNA to bind nanomaterials into organized 1D groupings, 2D scaffolds, or in 3D assembled crystals, has revealed the strength of using biomolecular tunability, flexibility, and mechanical rigidity in the organization of monomeric building-blocks. However, the design and fabrication of well-defined clusters, containing 2-10 nanoparticles apiece, for example, with controlled anisotropy, high yields, and under high concentrations, has remained elusive. The investigation of controlled nanoparticle clustering with DNA was pioneered by Alivisatos and co-workers, who have elegantly utilized nanoparticles with a precise number (˜1) of DNA attached. This approach has led to well-defined clusters, whose morphologies mimic the DNA scaffold motif used. In addition, such quantized groupings allow for advanced structure-function studies related to surface enhanced Raman spectroscopy (SERS), surface plasmon phenomenology, and metal enhanced fluorescence (MEF). Despite the strength of this scaffolding approach, the necessity for preparation and purification of mono-functionalized DNA-particles, as well as assembled product purification, has many shortcomings with respect to fabrication yields, simplicity, and modularity.

Scheme 1 in FIG. 1 illustrates the current state of the art in the assembly of nanoparticle clusters or groupings, such as “dimers.” First, gold nanoparticles are functionalized with a low coverage of DNA, which consists of ˜1-5 DNA/Au. Second, the particles are separated from one another based on either charge or size, which is related to the number of DNA/Au. This step is labor intensive, especially in extraction, and results in low yields. Finally, particles with an estimated ˜1 DNA/Au are combined with their complementary particles (i.e., particles A & B), which have also been extensively purified. In the next step, the particles are allowed to assemble in dilute conditions, and the assembly products are again purified, which results in again lower product yields.

Diagram 1 in FIG. 2 is a basic flow sheet describing the current state of the art process for making a solution consisting mostly of dimers. Particles A, generally but not always gold, are functionalized with DNA in dilute solution resulting in about 1 to 5 DNA per nanoparticle (Step K). The resulting solution is purified using electrophoresis (Step L), which generally takes hours and leaves behind impurities acquired during the process itself. Functionalized particles are then sorted (Step M), by size or by charge, to separate monomers, those nanoparticles functionalized by a single DNA molecule, from other particles (multimers, such as dimers, trimers, etc.). Multimers are discarded (Step N) and the resulting solution is again purified with electrophoresis (Step O), adding hours to the process, reducing yield, and adding impurities. The same process is applied to particles B (Steps P through T). The two types of particles having exactly one DNA per particle are combined (Step U), and electrophoretically purified (Step V). The resulting aggregates are assembled in dilute solution (Step W) and again purified (Step X), completing the synthesis (Step Y). In all, at least six electrophoretic purification steps are required in this process, each significantly reducing yield, introducing impurities, and consuming time.

Despite the initial advances in this type of assembly with mono-functionalized particles, present forms have a number of limitations that deleteriously affect their full scale adoption and commercial viability. A few of these limitations involve present approaches to controlling nanoparticle assembly into quantized groupings, requiring extensive purification steps of both monomeric building blocks and intermediate and final products. This results in low final yields (number of groupings), as well as introducing impurities in the system as a result of separation via gel electrophoresis and liquid chromatography which, while separating singletons from groupings of multiple particles, introduce impurities from the techniques themselves into the batch of singletons.

### SUMMARY

The present invention overcomes many of the limitations of the prior art. The invention is a first of its kind solid-state assembly/disassembly route. Using encoded solid supports, particles are assembled/disassembled and functionalized in an easily controllable and high yield approach which utilizes biomolecules (e.g., DNA, RNA, peptides) to encode interactions and anisotropy.

The present invention has several advantages over the prior art including: designing a general approach towards controlled anisotropy in nano-scale systems, where, for example, particular binding interactions (such as with DNA) are confined to particular areas of the particles; designing a general approach which illustrates the ability to self-assemble “Janus”-like morphologies, where each hemisphere of a nanoparticle contains different interactions or functionalization; designing and implementing a high throughput approach, which can be easily scaled, and which contains a “plug and play” modularity which may lead to the introduction of multiple classes of materials and interactions.

In particular, the nanoclusters and the methods of making the nanocluster (i.e., “Nano-Assembly platform using Encoded Solid Supports (NAESS)”) assembles nanomaterials at a colloidal substrate in a layer-by-layer fashion in which interactions between layers have been encoded using biomolecules, allowing for controlled interactions, purification of side products, modularity, and construction of complex architectures. In addition, the approach allows fabrication of nanoparticles with anisotropic interactions, Janus particles.

The invention improves the ability to impart anisotropy in nanoscale assembly systems using a solid-state assembly approach. It results in: 1) DNA-addressed specificity between particles or DNA layers with solid support; 2) a solid support easily derivatized for particle assembly and disassembly; and 3) interactions driven by biomolecular interactions, and assembly and dis-assembly requiring no change to environmental conditions, including, pH change, temperature change, buffer change, and radiation exposure.

In the present invention, planar or colloidal surfaces are grafted with biomolecules, which allows for a specific immobilization of corresponding bio-encoded nano-objects. A cluster can thus be built via the sequential attachment of recognition encoded components from the solution. Such components include bio-functionalized nano-objects or biomolecular linkers, which allows for the ability to control interparticle distances and cluster geometry.

In one embodiment, the present invention provides a method of making a nanocluster. The method comprises providing a surface comprising at least one anchoring biomolecule, wherein the surface is in a solution; adding an initial recognition-nano-component to the solution wherein the initial recognition-nano-component comprises i) a nanoparticle and one specifically-bindable-biomolecule, or ii) a nanoparticle and two different types of specifically-bindable-biomolecules, wherein a biomolecule of the initial recognition-nano-component specifically binds to the anchoring biomolecule; and adding a releasing biomolecule to the solution, wherein the releasing biomolecule binds to the anchoring biomolecule with a greater binding strength than the anchoring biomolecule binds to the initial recognition-nano-component, or wherein the releasing biomolecule binds to the initial recognition-nano-component with a greater binding strength than anchoring biomolecule binds to the initial recognition-nano-component, thereby making a nanocluster.

In some embodiments, the method further comprises (a) providing a plurality of recognition-nano-components, wherein a recognition-nano-component comprises i) a specifically-bindable-nanoparticle, ii) a nanoparticle and one specifically-bindable-biomolecule, or iii) a nanoparticle and two different types of specifically-bindable-biomolecules; (b) adding a recognition-nano-component to the solution, wherein the recognition nano-component specifically binds to a biomolecule of the initial recognition-nano-component; (c) subsequently adding a recognition-nano-component to the solution, wherein the recognition nano-component specifically binds to a biomolecule of most recently added recognition-nano-component of the nanocluster. Step (c) is repeated until a desired number of recognition-nano-components are sequentially specifically bonded to the nanocluster.

In some embodiments, the method further comprises adding a series of capping-moieties to the solution wherein the capping-moieties specifically bind to the unreacted biomolecules of the nanocluster except for the biomolecule of the most recently added recognition-nano-component of the nanocluster.

In some embodiments, the method further comprises adding an isolating surface to the solution wherein the isolating surface specifically binds to the unreacted biomolecules of the most recently added recognition-nano-component of the nanocluster, and washing away unreacted biomolecules.

In some embodiments, the method further comprises purifying the solution before a recognition-nano-component is added.

The nanocluster can comprise about two to about one hundred recognition-nano-components. A typical nanocluster comprises two recognition-nano-components.

The nanocluster can comprise a metal nanoparticle, a semiconductor nanoparticle, an organic nanoparticle, silica, or combinations thereof. Preferably, the metal nanoparticle is a gold nanoparticle, a silver nanoparticle, a copper nanoparticle, a platinum nanoparticle or a palladium nanoparticle.

In some embodiments, the method further comprises adding a linker to the solution before adding a recognition nano-component, wherein the linker specifically binds to a biomolecule on each of two sequentially added recognition-nano-components, thereby attaching a linker between the two sequentially added recognition-nano-components. The linker can be added so that the approximate ratio of a linker to a recognition-nano-component is about 1:1 to about 10:1. In a preferred embodiment, the linker is added so that the approximate ratio of a linker to a recognition-nano-component is about 5:1.

The specifically-bindable biomolecules of the nanocluster can comprise single-stranded nucleic acid molecules; antigens; moieties that bind antigens; or combinations thereof. Typically, the single-stranded nucleic acid molecules comprises about six to about 200 bases, or about ten to about thirty bases.

In one embodiment of the present invention, a method of detecting the presence of a particular target biomolecule with a dimer in a sample is provided. The method comprises (a) providing a detection dimer, wherein the detection dimer comprises a first recognition nano-component attached to a second recognition nano-component, wherein the first recognition nano-component comprises a first nanoparticle and a first specifically-bindable biomolecule, wherein the second recognition nano-component comprises a second nanoparticle and a second specifically-bindable biomolecule. In one embodiment, the first recognition nano-component is attached to the second recognition nano-component by binding of the first biomolecule to the second biomolecule, wherein the first biomolecule binds to the second biomolecule with an initial binding strength. In another embodiment, the first recognition nano-component is attached to the second recognition nano-component by a linker which binds the first biomolecule to the second biomolecule, wherein the linker binds the first biomolecule to the second biomolecule with an initial binding strength. A sample is contacted with the detection dimer. If the target biomolecule is present in the sample, then (i) the target biomolecule binds to either the first biomolecule or the second biomolecule with a detection binding strength, wherein the detection binding strength is greater than the initial binding strength; or (ii) the target biomolecule binds to the linker with a detection binding strength, wherein the detection binding strength is greater than the initial binding strength. It is determined whether the first recognition nano-component became detached from the second recognition nano-component to form monomers, wherein if monomers were formed to a sufficient level, then the target biomolecule is present.

FIGS. 17 to 23 show both nucleic acid detection and protein antigen detection using the detection dimers of the present invention.

In another embodiment of the present invention, a method of detecting the presence of a particular target biomolecule with a trimer in a sample is provided. The method comprises (a) providing a detection trimer. The detection trimer comprises a first recognition nano-component attached to a second recognition nano-component and a third recognition nano-component, wherein the first recognition nano-component comprises a first nanoparticle and a first single strand nucleic acid molecule, wherein the second recognition nano-component comprises a second nanoparticle and a second single strand nucleic acid molecule, and wherein the third recognition nano-component comprises a third nanoparticle and a third single strand nucleic acid molecule, wherein the first recognition nano-component is attached to the second recognition nano-component by a single stranded nucleic acid linker which binds i) the portion of the first nucleic acid molecule which is more proximate to the first nanoparticle to ii) the second nucleic acid molecule, wherein the linker binds the first nucleic acid molecule and the second nucleic molecule with an initial binding strength; wherein the first recognition nano-component is attached to the third recognition nano-component by the binding of i) the portion of the first nucleic acid molecule which is more distal to the first nanoparticle to ii) the third nucleic acid molecule, wherein the first nucleic acid molecule and the third nucleic molecule bind with an initial prime binding strength. The sample is contacted with the detection trimer. If the first target biomolecule is present in the sample, the first target biomolecule binds either the portion of the first nucleic acid molecule which is more proximal to the first nanoparticle or the second nucleic acid molecule or the linker with a detection binding strength, wherein the detection binding strength is greater than the initial binding strength; and (ii) wherein if the second target biomolecule is present in the sample the second target biomolecule binds either the portion of the first nucleic acid molecule which is more distal to the first nanoparticle or the third nucleic acid molecule with a detection prime binding strength, wherein the detection prime binding strength is greater than the initial prime binding strength. It is then determined whether the first recognition nano-component became detached from the second recognition nano-component and/or third recognition nano-component to form dimers and/or monomers, wherein if dimers and/or monomers were formed to a sufficient level, then the first target biomolecule and/or second target biomolecule is present.

The present method provides for the rational design and fabrication of nanoclusters using bio-encoded nanoscale building blocks in a stepwise assembly and release at a solid substrate. Using programmable recognition biomolecules as a nanoparticle encoding motif, the nanoparticles have been imparted with anisotropy for both the assembly and disassembly at a colloidal substrate, which allowed for the fabrication of well defined multi-particle clusters and Janus nanoparticles, with remarkably high fidelity and yields. In addition, the described method is highly modular, which allowed for ease of incorporation of different nano-components for the assembly of systems with regulated optical properties, thus, demonstrating its versatility for fabrication of designed nanomaterials.

### BRIEF DESCRIPTION OF THE DRAWINGS

FIG. 1: Scheme 1: Current state of the art in the assembly of nanoparticle clusters with well defined assembly morphology. Each step requires labor intensive purification via gel electrophoresis or size exclusion chromatography. In addition, each step is accompanied by a successive decrease in product yield, which also requires dilute assembling conditions.

FIG. 2: Diagram 1 in is a basic flow sheet describing the current state of the art process for making a solution consisting mostly of dimers.

FIG. 3: Diagram 2 is a simple view of the process of the present invention.

FIG. 4: Scheme 2: A generalized scheme of the Nano-Assembly platform using Encoded Solid Supports (NAESS) approach; each layer is formed via biomolecular or chemical recognition.

FIG. 5: Scheme 3: A schematic illustration of the nano-assembly platform using encoded solid supports (NAESS) approach.

FIG. 6: A typical TEM micrograph of the released Au dimers fabricated by the stepwise NAESS method. Image collected after ˜1 day release and without further purification. Statistical analysis of the assembled dimers reveals ˜72% dimers, 26.7% monomers, and only 1.3% of structures with 3 or more particles after counting 1670 particles.

FIG. 7: The use of the solid-state assembly approach for creation of anisotropic assembly and cluster formation. Large DNA-capped Au (˜50 nm) are assembled and functionalized identically to steps 1-2 (Scheme 1). The anisotropic Janus-like particle is released from the 1-Mag, and mixed with complementary capped Au (10 nm). The result (a-c), as shown via TEM is the selective binding of the small Au cores to only one side of the large particles.

FIG. 8: A collection of UV-vis results for multiple doublet systems, including 10 nm Au dimers assembled via complementary DNA-capping (A), 22 nm Au dimers (B), and Ag—Au hetero-dimers using 23 nm Ag and 10 nm Au, with accompanying TEM micrographs of the specific samples studied (insert).

FIG. 9: Comparison of Prior Art Architectured Nanoclusters and Nanoclusters of Present Invention.

FIG. 10: Hybrid Structures for Energy/Charge Transfer.

FIG. 11: An idealized illustration of the assembly and release of nanoparticle dimers or Janus particle monomers at an encoded support.

FIG. 12: A typical TEM micrograph demonstrating the fabrication of dimers.

FIG. 13 (A) shows a corresponding set of DLS (a) and UV-vis (b) results for isolated 75 nm C—Au and assembled dimers. FIG. 13 (B) shows a comparison between the observed SP-band shift (Δλ/λ₀), and interparticle distance to particle diameter ratio (d/D), with exponential decay fit via y=C₁exp(−(x/τ))+y₀, where τ=0.14±0.028, A=0.06±0.005, with TEM micrograph sampled from each studied solution. (C) A corresponding set of DLS (a) and UV-vis (b) results for 27 nm A′/B—Ag and 11 nm C—Au monomers, and their assembled hetero-dimer, with a TEM micrograph sampled from the studied solution.

FIG. 14: A set of TEM micrographs from three independent dimer fabrication trials (A-C), with statistical analysis of dimer yield, revealing; 83%, 79%, and 70% dimers for samples A-C respectively. C: TEM micrographs sampled from typical concentration (˜8 nM) (a), and after dilution to ˜0.5 nM.

FIG. 15: A set of DLS (a) and normalized UV-vis spectra (b) characterizing the Dₕ and SP-band characteristics of isolated C—Au monomers, and assembled dimers for 11 nm Au (A), 20 nm Au (B), and 50 nm Au (C). (0.01 M Phosphate Buffer, 0.1M NaCl, pH=7.4).

FIG. 16: Characterization of Au monomers, and assembled dimers via agarose gel electrophoresis (1% agarose, 80 mV, 0.5×TBE, 10% Ficoll 400).

FIG. 17: Detection Concept Using Dimer Nano-clusters.

FIG. 18: DNA Detection Using Designed Dimer Nano-clusters.

FIG. 19: Building Dimer Nano-clusters.

FIG. 20: Optical Properties of Dimer Nanoclusters.

FIG. 21: DNA Detection using the Nanocluster Biosensors.

FIG. 22: Target DNA Detection: Kinetics.

FIG. 23: Target DNA Detection: Mismatches.

FIG. 24: Nucleic Acid Detection Trimer.

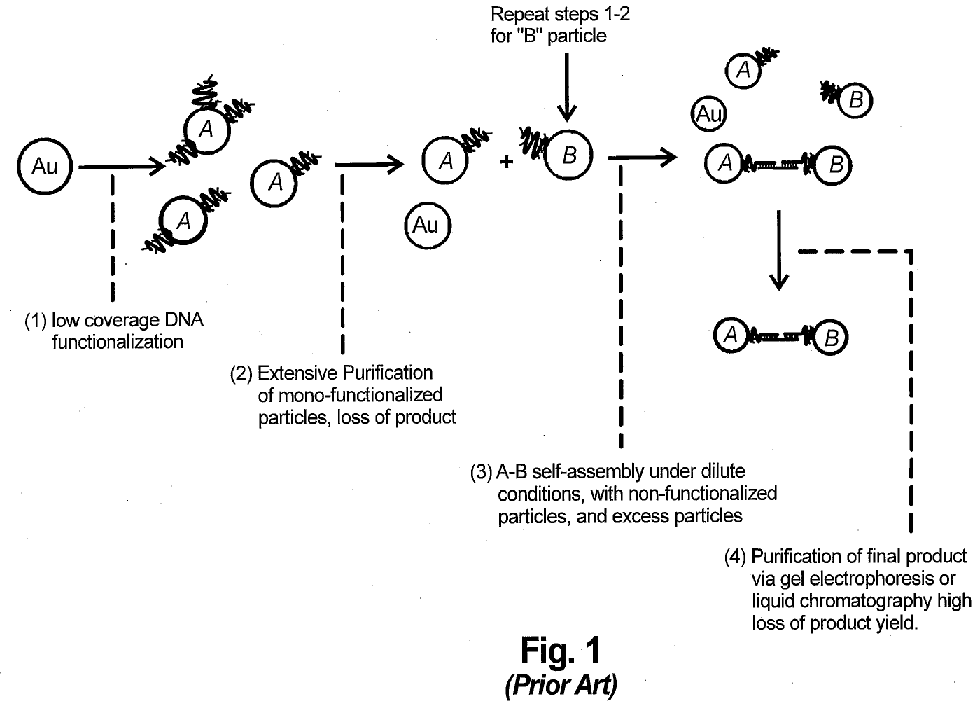

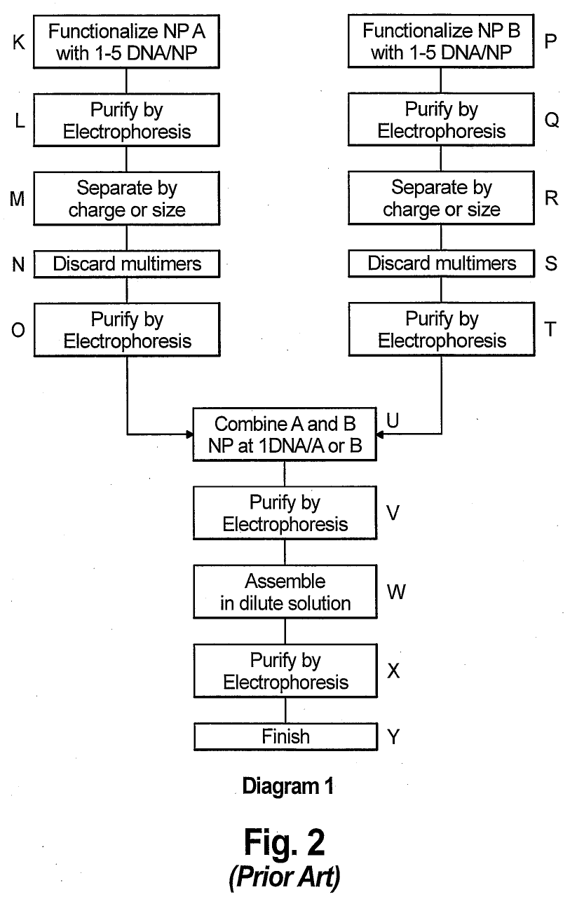

### DETAILED DESCRIPTION OF THE INVENTION

In one aspect, the invention provides a highly controllable method of making nanoclusters. A nanocluster is made by sequentially adding nanocluster “building blocks” by which the nanocluster is self-assembled layer by layer. This method takes place in solution with a growing nanocluster immobilized on a solid support. The “building blocks” of a nanocluster are termed “recognition-nano-components.”

### Recognition-Nano-Components

A “recognition-nano-component” is a nanoparticle with the ability to specifically bind one or two types of biomolecules. The nanoparticle has the ability to specifically bind biomolecules due to i) having one or two specifically bindable-biomolecules attached to its surface, or ii) its intrinsic ability to bind affinity tags. By means of this specific binding, recognition-nano-components of different types (i.e., different species) can precisely be attached to one another.

In particular, a recognition-nano-component of the present invention comprises (or consists essentially of): i.) a nanoparticle and one attached specifically-bindable-biomolecule, ii.) a nanoparticle and two different types of attached specifically-bindable-biomolecules, wherein the two different types of attached specifically-bindable-biomolecules have low affinity for one another, or iii.) a specifically-bindable-nanoparticle.

A description of the specific components of recognition-nano-components follows.

### Nanoparticles

In this specification, a “nanoparticle” refers to a metal particle, semiconductor particle, organic particle or silica particle with a diameter in the nanometer (nm) range. Preferably, the nanoparticle has a minimum diameter of about 1 nm and a maximum diameter of about 100 nm, more typically from about 2 nm to about 50 nm.

The metal nanoparticle can be any metal, metal oxide, or mixtures thereof. Some examples of metals useful in the present invention include gold, silver, platinum, and copper. Examples of metal oxides include iron oxide, titanium oxide, chromium oxide, cobalt oxide, zinc oxide, copper oxide, manganese oxide, and nickel oxide.

The metal or metal oxide can be magnetic. Examples of magnetic metals include, but are not limited to, iron, cobalt, nickel, manganese, and mixtures thereof. An example of a magnetic mixture of metals is a mixture of iron and platinum. Examples of magnetic metal oxides include, for example, iron oxide (e.g., magnetite, hematite) and ferrites (e.g., manganese ferrite, nickel ferrite, or manganese-zinc ferrite).

The semiconductor nanoparticle is capable of emitting electromagnetic radiation upon excitation. Some examples of semiconductors include Group II-VI, Group III-V, and Group IV semiconductors. The Group II-VI semiconductors include, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, and mixtures thereof. Group III-V semiconductors include, for example, GaAs, GaN, GaP, GaSb, InGaAs, InP, InN, InSb, InAs, AlAs, AlP, AlSb, AlS, and mixtures therefore. Group IV semiconductors include, for example, germanium, lead, and silicon.

The semiconductor can also include mixtures of semiconductors from more than one group, including any of the groups mentioned above.

The semiconductor nanoparticles used in the invention preferably have the capability of absorbing radiation over a broad wavelength band. The wavelength band includes gamma radiation to microwave radiation.

The semiconductor nanoparticles preferably have the capability of emitting radiation within a narrow wavelength band of about 40 nm or less, preferably about 20 nm or less. A narrow emission band permits the simultaneous use of a plurality of differently colored semiconductor nanoparticle complexes with different semiconductor nanoparticles without overlap (or with a small amount of overlap) in wavelengths of emitted light when exposed to the same energy source.

The frequency or wavelength of the narrow wavelength band of light emitted from the semiconductor nanoparticle may be further selected according to the physical properties, such as size, of the semiconductor nanoparticles.

In a preferred embodiment, the semiconductor nanoparticle is fluorescent. The fluorescence of the semiconductor nanoparticle is preferably preserved (e.g., is not quenched).

Organic nanoparticles comprise mainly organic materials such as polymers (e.g., polystyrene) or complexes of smaller organic molecules. Examples of organic nanoparticles include liposomes, dendrimers, carbon nanomaterials and polymeric micelles. Liposomes are phospholipid vesicles (50-100 nm) that have a bilayer membrane structure similar to that of biological membranes and an internal aqueous phase. Dendrimers are highly branched synthetic polymers (<15 nm) with layered architectures constituted of a central core, an internal region and numerous terminal groups that determine dendrimer characteristics. Carbon nanomaterials include fullerenes in the form of a hollow sphere, ellipsoid, tube, or plane. Carbon nanotubes are formed of coaxial graphite sheets (<100 nm) rolled up into cylinders. Examples of polymeric micelles include polystyrene beads.

Additionally, polymers can be incorporated into silica to form silica semiconducting polymer nanocomposites.

Specifically-Bindable Biomolecules

Various types and classes of specifically-bindable biomolecules are attached to nanoparticles to form the recognition nano-components of the present invention. Examples include, for instance, base-pairing nucleic acids, and other moieties.

### Base-Pairing Nucleic Acids

The following discussion describes base-pairing nucleic acids used in recognition-nano-components of the present invention.

A nucleic acid is a macromolecule composed of chains of monomeric nucleotides. Nucleotides consist of three joined structures: a nitrogenous base, a sugar, and a phosphate group.

The nitrogenous bases can be any naturally-occurring purines and pyrimidines or modified purines and pyrimidines. Typically, the bases of the present invention are adenine, guanine, cytosine, thymidine and uracil.

The bases can be modified, for example, by the addition of substituents at one or more positions on the pyrimidines and purines. The addition of substituents may or may not saturate any of the double bonds of the pyrimidines and purines. Examples of substituents include alkyl groups, nitro groups, halogens and hydrogens. The alkyl groups can be of any length, preferably from one to six carbons. The alkyl groups can be saturated or unsaturated; and can be straight-chained, branched or cyclic. The halogens can be any of the halogens including, bromine, iodine, fluorine or chlorine.

Further modifications of the bases can be the interchanging and/or substitution of the atoms in the bases. For example, the positions of a nitrogen atom and a carbon atom in the bases can be interchanged. Alternatively, a nitrogen atom can be substituted for a carbon atom; an oxygen atom can be substituted for a sulfur atom; or a nitrogen atom can be substituted for an oxygen atom.

Another modification of the bases can be the fusing of an additional ring to the bases, such as an additional five or six-membered ring. The fused ring can carry various further groups.

Specific examples of modified bases include 2,6-diaminopurine, 2-aminopurine, pseudoisocytosine, E-base, thiouracil, ribothymidine, dihydrouridine, pseudouridine, 4-thiouridine, 3-methlycytidine, 5-methylcytidine, inosine, N⁶ methyladenosine, N⁶ isopentenyladenosine, 7-methylguanosine, queuosine, wyosine, etheno-adenine, etheno-cytosine, 5-methylcytosine, bromothymine, azaadenine, azaguanine, 2′-fluoro-uridine and 2′-fluoro-cytidine.

The bases are attached to a molecular backbone. The backbone comprises sugar or non-sugar units. The units are joined in any manner known in the art.

In one embodiment, the units are joined by linking groups. Some examples of linking groups include phosphate, thiophosphate, dithiophosphate, methylphosphate, amidate, phosphorothioate, methylphosphonate, phosphorodithioate and phosphorodiamidate groups.

Alternatively, the units can be directly joined together. An example of a direct bond is the amide bond of a peptide.

The sugar backbone can comprise any naturally-occurring sugar. Examples of naturally-occurring sugars include ribose and deoxyribose, for example 2-deoxyribose.

The sugars of the backbone can be modified in any manner. Examples of modified sugars include 2′-O-alkyl ribose, such as 2′-O-methyl ribose and 2′-O-allyl ribose. Preferably, the sugar units are joined by phosphate linkers. The sugar units may be linked to each other by 3′-5′, 3′-3′ or 5′-5′ linkages. Additionally, 2′-5′ linkages are also possible if the 2′ OH is not otherwise modified.

The non-sugar backbone can comprise any non-sugar molecule to which bases can be attached. Non-sugar backbones are known in the art.

In one embodiment, the non-sugar backbone comprises morpholine rings (tetrahydro-1,4-oxazine). The resulting base-pairing segment is known as a morpholino oligo. The morpholine rings are preferably joined by non-ionic phosphorodiamidate groups. Modified morpholines known in the art can also be used in the present invention.

In another embodiment, the non-sugar backbone comprises modified amino acid units linked by, for example, amide bonds. The amino acids can be any amino acid, including natural or non-natural amino acids. The amino acids can be identical or different from one another. The amino acids are preferably amino alkyl-amino acids, such as (2-aminoethyl)-amino acid.

Bases are attached to this backbone by molecular linkages. Examples of linkages are methylene carbonyl, ethylene carbonyl and ethyl linkages. The resulting pseudopeptide is known as a peptide nucleic acid (PNA). (Nielsen et al., Peptide Nucleic Acids-Protocols and Applications, Horizon Scientific Press, pages 1-19; Nielsen et al., Science 254: 1497-1500.)

A preferred example of a PNA is N-(2-aminoethyl)-glycine. Further examples of PNAs include cyclohexyl PNA, retro-inverso, phosphone, propionyl and aminoproline PNA.

Other examples of artificial nucleic acids include locked nucleic acid (LNA), glycol nucleic acid (GNA) and threose nucleic acid (TNA). Each of these is distinguished from naturally-occurring DNA or RNA by changes to the backbone of the molecule.

The single-stranded nucleic acid molecules used as the biomolecules of the recognition-nano-components comprise about six to about 200 bases, about ten to about thirty bases, or about fifteen to twenty bases.

### Specifically-Bindable Biomolecules Other Than Nucleic Acids

Various types of biomolecules, other than nucleic acids, can be attached to nanoparticles to form the recognition nano-components of the present invention. Examples of specifically binding moieties, other than nucleic acids, include receptors and their ligands, antibodies and their antigens (both protein and non-protein antigens), and protein affinity tags and moieties recognized by the tags.

Antibodies include whole antibodies, functional equivalents thereof, monoclonal antibodies, and functional equivalents derived from monoclonal antibodies.

Suitable equivalents of antibodies include any fragment that comprises a sufficient portion of the hypervariable region to bind specifically to an antigen. Such fragments may, for example, contain one or both Fab fragments, or the F(ab′)₂ fragment. Preferably, the antibody fragments contain all six complementarity determining regions of the whole antibody, although functional fragments containing fewer than all of such regions, such as three, four or five CDRs, may also be suitable. The preferred fragments are single chain antibodies, or Fv fragments.

Examples of antibodies are members of any class of immunoglobulins, such as: IgG, IgM, IgA, IgD, or IgE, and the subclasses thereof. The preferred antibodies are members of the IgG1 subclass.

A preferred example of biomolecule is coxsackievirus-adenovirus receptor (CAR) D1 domain which serves as a receptor for group B human coxsackievirus and many adenovirus serotypes. Examples of ligands which are suitable as biomolecules include ligands of the aforementioned immunoglobulins. A preferred example of a ligand is the knob protein domain of an adenovirus. Examples of serotypes of adenovirus include serotype 2, serotype 5 and serotype 12.

A receptor-ligand pair is another example of a biomolecule pair suitable for the invention. Ligands can be natural or synthetic molecules, such as hormones (e.g., gastrointestinal peptidic hormones) or neurotransmitters, which specifically bind to a receptor. Some examples of receptor-specific ligands include bombesin and transferrin.

Other examples include cytokine receptors, e.g., interleukin-1 receptor type I, interleukin-1 receptor type II precursor (IL-1R-2, IL-1R-beta, CD121b antigen), platelet-derived growth factor receptor (PDGFR), interleukin-6 receptor alpha chain precursor (IL-6R-alpha,CD126 antigen), macrophage colony-stimulating factor 1 receptor precursor (CSF-1-R, CD115 antigen), mast/stem cell growth factor receptor precursor (SCFR, c-kit, CD117 antigen), basic fibroblast growth factor receptor 1 precursor (FGFR-1, Tyrosine kinase receptor CEK1), vascular endothelial growth factor (VEGF) receptor, and epidermal growth factor (EGF) receptors, e.g., HER-1, HER-2, HER-3, and HER-4. Another example of a biomolecule pair suitable for the invention is biotin-avidin.

### Specifically-Bindable-Nanoparticles

A specifically-bindable-nanoparticle is a nanoparticle which can specifically bind to a biomolecule, for example, can specifically bind to an affinity tag. Affinity tags can be generated against the surfaces of metallic particles. For instance, a poly-histidine tag can be used to bind to a nickel-nitrilotriacetic acid (Ni-NTA) modified particle. Generation of such specifically-bindable-nanoparticle is known in the art. See Table I acquired from Sarikaya et al., “Molecular Biomimetics: Nanotechnology through Biology,” Nature Materials 2:577-585 (2003).

TABLE I

Examples of polypeptide sequences
exhibiting affinity for various inorganics.

| | | | | | | |
|---|---|---|---|---|---|---|
| Materials | Sequences | Size | pl^{a} | MW^{b} | Charge^{c} | Display^{Ref} |

| | | | | | | |
|---|---|---|---|---|---|---|
| Au | MHGKTQATSGTIQS | 10 | 8.52 | 1446.60 | +1 | CSD^{29,43} |
| | SKTSLGQSGASLQGSEKLTNG | 21 | 8.31 | 2050.21 | +1 | |
| | QATSEKLVRGMEGASLHPAKT | 21 | 8.60 | 2211.52 | +1 | |
| Pt | DATSTWR | 7 | 9.60 | 920.98 | +1 | PD^{d} |
| | QSVTSTK | 7 | 8.75 | 749.82 | +1 | |
| | SSSHLNK | 7 | 8.49 | 771.83 | +1 | |
| Pd | SVTQNKY | 7 | 8.31 | 838.92 | +1 | PD^{d} |
| | SPHPGPY | 7 | 6.46 | 753.81 | 0 | |
| | HAPTPML | 7 | 6.74 | 765.93 | 0 | |
| Ag | AYSSGAPPWPPF^{e} | 12 | 5.57 | 1221.89 | 0 | PD⁵⁴ |
| | NPSSLFRYLPSD^{e} | 12 | 6.09 | 1395.53 | 0 | |
| | SLATQPPRTPPV^{e} | 12 | 9.47 | 1264.46 | +1 | |
| SiO₂ | MSPHFHPRHHHT^{e} | 12 | 9.59 | 1470.63 | +1 | PD⁴⁵ |
| | RGRRRRLSCRLL^{e} | 12 | 12.30 | 1541.89 | +6 | |
| | KPSHHHHHTGAN | 12 | 8.78 | 1359.43 | +1 | |
| Zeolites | VKTQATSREEPPRLPSKHRPG | 21 | 10.90 | 2371.68 | +3 | CSD⁵⁹ |
| | MDHGKYRQKQATPG | 14 | 9.70 | 1616.82 | +2 | |
| ZnO | NTRMTARQHRSANHKSTQRA^{e} | 20 | 12.48 | 2351.59 | +4 | CSD⁵⁶ |
| | YDSRSMRPH | 9 | 8.75 | 1148.26 | +1 | |
| CaCO₃ | HTQNMRMYEPWF | 12 | 6.75 | 1639.87 | 0 | PD⁵⁸ |
| | DVFSSFNLKHMR | 12 | 8.75 | 1480.70 | +1 | |
| Cr₂O₃ | VVRPKAATN | 9 | 11.00 | 955.13 | +2 | CSD⁵⁹ |
| | RIRHRLVGO | 9 | 12.30 | 1134.35 | +3 | |
| Fe₂O₃ | RRTVKHHVN^{e} | 9 | 12.01 | 1146.32 | +3 | CSD⁶⁰ |
| GaAs | AQNPSDNNTHTH | 12 | 5.97 | 1335.31 | 0 | PD⁴⁴ |
| | RLELAIPLQGSG | 12 | 6.00 | 1253.46 | 0 | |
| | TPPRPIQYNHTS | 12 | 8.44 | 1410.55 | +1 | |
| ZnS | NNPMHQN^{e} | 7 | 6.74 | 853.91 | 0 | PD⁵⁵ |

^{a}Isoelectric points and ^{b}Molecular masses of peptides are calculated using Compute pl/Mw tool (http://us.expasy.org/tools/pl_tool.html).
^{c}Calculated by subtracting the number of basic residues (R and K) from the number acidic residues (D and E).
^{d}Unpublished by the authors.
^{e}Most frequently observed sequences.

### Method of Making Nanoclusters

In the method, a surface (i.e., a solid support) having at least one anchoring biomolecule, typically a plurality of anchoring biomolecules, is provided. An anchoring biomolecule is a specifically-binding biomolecule as described above. Preferably the anchoring biomolecule is a nucleic acid.

The surface is any type of solid support such as, for example, planar surfaces and colloidal surfaces. Examples of planar surfaces include silicon wafers and polystyrene microtiter plates. Examples of colloidal surfaces include magnetic beads. The surface is in a solution, such as an aqueous buffer solution, made by methods known in the art.

A first recognition nano-component, termed “initial recognition nano-component, is then added to the solution.” The initial recognition-nano-component comprises a nanoparticle and two different types of specifically-bindable-biomolecules wherein one biomolecule specifically binds to the anchoring biomolecule.

Different recognition-nano-components are then sequentially added to the solution in a series of steps. In each step, one species (i.e., type) of recognition-nano-component is added. In the first step, a recognition nano-component is added that specifically binds to a biomolecule of the initial recognition-nano-component. Subsequently, different species of recognition-nano-components are sequentially added to the solution, wherein at each step, a particular recognition nano-component specifically binds to the biomolecule of the most recently added recognition-nano-component of the nanocluster. The nanocluster can be thought of as a structure with different “layers” of recognition nano-components. The most recently added recognition-nano-component of the nanocluster can be thought of as the “top layer” of the nanocluster.

Due to geometric constraints and steric hindrance between the biomolecules within one species of biomolecules, the linking of additional recognition-nano-components occurs only on the top hemisphere of the nanoparticles, which results in anisotropically functionalized clusters. This feature enables the ability of the nanoclusters to self-assemble.

Some of the biomolecules within a nanocluster may remain unreacted. Accordingly, in some embodiments, the method further comprises adding a series of capping moieties to the solution. The series of capping moieties specifically bind to each species of unreacted biomolecules within each layer but are otherwise essentially inert. Alternatively, in some embodiments, capping moieties are added for each species of unreacted biomolecule except for the biomolecules of the top layer.

Recognition nano-components are added sequentially until a desired number of layers make up the nanocluster. Once a desired number of layers is obtained, a releasing biomolecule (i.e., fuel biomolecule) is added to the solution to release the nanocluster from the surface. The releasing biomolecule binds to the anchoring biomolecule with a greater binding strength than the anchoring biomolecule binds to the initial recognition-nano-component, or the releasing biomolecule binds to the initial recognition-nano-component with a greater binding strength than anchoring biomolecule binds to the initial recognition-nano-component, thereby releasing the one-component nanocluster.

The number of layers of recognition nano-components in a nanocluster is not critical. Typically, the nanocluster comprises about two to about one hundred layers or about five to about fifty layers. A nanocluster comprising two layers of recognition-nano-components is termed a “dimer.” If a one component nanocluster is desired, then the nanocluster is released from its surface after the binding of the initial recognition-nano-component.

Nanoclusters which have an unreacted layer have anisotropy, and thus are Janus nanoclusters. One manner by which to obtain a Janus nanocluster is by adding the capping moieties for each species of unreacted biomolecule except for the biomolecules of the top layer, and having the releasing biomolecule bind to the initial recognition-nano-component. Another manner by which to obtain a Janus nanocluster is by adding the capping moieties for each species of unreacted biomolecule, and having the releasing biomolecule bind to the anchoring biomolecule.

The resulting nanocluster product can be isolated from the solution by several methods. The methods are based on contacting the solution with a surface or surfaces (i.e., “isolating surface(s)”) that specifically bind unreacted recognition-nano-components in the solution.

In one embodiment, unreacted recognition-nano-components (i.e., recognition-nano-components which did not bind to the nanocluster) are removed from the solution to isolate the resulting nanocluster. The unreacted recognition-nano-components can be removed before each step of adding a new species of recognition-nano-component to the solution, or the unreacted recognition nano-components can be removed after the nanocluster product has been assembled. In this embodiment, more than one isolating surface is necessary. In particular, since each surface would bind a different recognition-nano-component, as many isolating surfaces are needed as species of unreacted recognition-nano-components.

In a preferred embodiment, the top layer of recognition-nano-components of the nanocluster is left unreacted, and an isolating surface which specifically binds to the unreacted biomolecules is contacted with the solution and the isolating surface is then removed from the solution. In this embodiment, only one isolating surface is necessary.

Depending on the specific isolating surface used, isolation involves either surface removal (e.g. via centrifugation and removal of the aqueous solution) or separation of magnetic beads by magnetic field followed by rinsing and re-dispersion in a new solution. In cases where the synthesis is performed on a planar surface (e.g., microtiter plate), the solution can be removed directly, after which the wells are washed to remove any unreacted molecules.

The resulting nanocluster can comprises more than one type of nanoparticle. For example, the nanocluster can comprise one or more metal nanoparticles and one or more semiconductor nanoparticles. The metal and semiconductor nanoclusters can be of different types.

### Linkers

In some embodiments, the nanoclusters have linkers in between some or all of the species of recognition-nano-components. In these embodiments, the method of the invention further comprises adding a linker to the solution before adding a recognition nano-component. The linker specifically binds two sequentially added recognition-nano-component species.

The linkers used between two recognition-nano-component species depend upon what two species are being linked.

If the biomolecules of two recognition-nano-components are single stranded nucleic acids, then the linker that links the two recognition nano-components are single stranded nucleic acids. The linkers can include from about two to about 100 bases, more typically about five to about twenty bases, and most typically about eight to about fifteen bases.

If the two recognition-nano-components to be linked contain: i) two biomolecules other than single stranded nucleic acids; ii) one biomolecule other than single stranded nucleic acid and one specifically-bindable-nanoparticle; or iii) two specifically-bindable-nanoparticles, then the linker used is a “linker peptide.”

A linker peptide is a peptide comprising (or consisting essentially of) two recognition regions and separated by a spacer. Each recognition region specifically binds to one of the recognition nano-components to be linked.

The recognition region can be peptide that is specifically bindable to the biomolecules described above. Examples include peptide receptors (e.g., CAR, VEGF-receptors, EGF receptors, cytokine receptors), protein ligands (e.g., Knob protein), and antibodies (typically, the binding domain of an antibody).

If the recognition-nano-component to be linked is a specifically-bindable-nanoparticle, then the recognition region of the linker peptide is an affinity tag. The affinity tag directly binds to the nanoparticle, i.e., the affinity tag binds to the nanoparticle without the nanoparticle having an attached biomolecule. Affinity tags are described above.

The spacer of the linker peptide can be any sequence of amino acids of about two to about twenty-five amino acids; more typically, about four to about twenty amino acids; and most typically about six to about fifteen amino acids. Preferably, the amino acids are rich in serine and glycine since they provide a flexible structure. An example of a spacer sequence is Gly-Gly-Ser-Gly-Gly-Ser-Gly-Gly.

An example of a linker peptide follows: A linker peptide consisting essentially of a first affinity tag which directly binds to the surface of nanoparticle 1 (e.g. a gold particle), a spacer sequence of six to fifteen amino acids rich in serine and glycine, and a second affinity tag which directly binds to the surface of nanoparticle 2 (e.g., a silver nanoparticle).

Another example of a linker peptide follows: A linker peptide consisting essentially of a first affinity tag which directly binds to the surface of a quantum dot, a flexible spacer sequence, followed by the CAR protein. In some embodiments, the linker peptide can be a recombinant CAR protein that has the affinity tag region plus the spacer sequence fused to either its C-terminal or N-terminal end. Such a peptide can be made with standard molecular biological techniques expressed under control of the T7 promoter.

In some embodiments, the two biomolecules of a recognition-nano-component are an antigen and a nucleic acid. In such case, on one hemisphere, the recognition-nano-component could be specifically bond to a DNA linker; and on the other hemisphere, the recognition-nano-component could be specifically bond to an antibody.

“Building blocks” (i.e., recognition-nano-components) of nanoclusters and various examples of nanoclusters follow,

### Building Blocks:

(ss-NP-ss)
(NP)
(NP-ss)
(BM-NP-ss)
(BM-NP-BM)
(BM-NP)
   wherein: NP=nanoparticle; BM=biomolecule; ss=single-stranded nucleic acid; and RR=Recognition Regions of a Peptide Linkers. Parenthesis indicates one “building block.”

Examples of Nanoclusters:

1. (Surface-ss)-(ss-NP)-(RR-spacer-RR)-(NP).

2. (Surface-BM)-(BM-NP)-(RR-spacer-RR)-(BM-NP).

3. (Surface-ss)-(ss-NP)-(RR-spacer-RR)-(BM-NP-ss)-(ss-NP).

4. (Surface-ss)-(ss-NP-ss)-(ss-spacer-ss)-(ss-NP-BM)-(BM-NP).

The amount of a linker added to the solution with respect to the amount of a recognition-nano-component can affect the structure of the resulting nanocluster. For example, to synthesize a dimer, the approximate ratio of a linker to a recognition-nano-component is typically about 1:1.

In some embodiments of the present invention, a plurality of “small” nanoparticles is placed on a “main nanoparticle.” These nanoclusters are termed “strawberry-like nanoclusters” or “strawberry structures.” In these embodiments, a linker is added so that the approximate ratio of a linker to a recognition-nano-component having the main nanoparticle is from about 2:1 to about 10:1. Then the small nanoparticle is added. If the small nanoparticle is much smaller than the main nanoparticle, then the relative amount of linker added determines the number of small nanoparticles that become attached to the main nanoparticle. However, the size of a small nanoparticle can also influence the number of small nanoparticles that can fit on a main nanoparticle.

In some embodiments, the main nanoparticle is about 2 nm to about 100 nm, and the small nanoparticles are about 4 nm to about 20 nm. Some illustrative examples of how the relative amount of linkers added and the relative size of the small nanoparticles influence the amount of small nanoparticles on the main nanoparticle follows.

Example: Main nanoparticle is about 50 nm. Linkers are added at ratio of 5× the main nanoparticles. Then 10 nm small nanoparticles are added. Result: about five small nanoparticle are attached to the main nanoparticles.

Example: Large nanoparticle is about 100 nm. Linkers are added at ratio of 5× the large nanoparticles. Then 20 nm small nanoparticles are added. Result: about five small nanoparticles are attached to the main nanoparticle.

Example: Large nanoparticle is about 100 nm. Linkers are added at ratio of 5× the large nanoparticles. Then 10 nm small particles are added. Result: about ten small nanoparticles are attached to the main nanoparticle.

Example: Large nanoparticle is about 100 nm. Linkers are added at ratio of 10× the large nanoparticles. Then 10 nm small particles are added. Result: about 10 small nanoparticles are attached to the main nanoparticle.

Example: Large nanoparticle is about 100 nm. Linkers are added at ratio of 10× the large nanoparticles. Then 2 nm small particles are added. Result: about 10 small nanoparticles are attached to the main nanoparticle.

Example: Large particle is about 100 nm. Linkers are added at ratio of 10× the large particles. Then 25 nm small particles are added. Result: about 5 small nanoparticles are attached to the main nanoparticle.

A typical nanoparticle of 10 nm has ˜50-60 DNA grafted to the surface. The amount depends on the diameter of a nanoparticle in quadratic way, as area. The amount of linkers attached to those DNA strands can be regulated, from 1 to ˜10-20

### Applications of the Nanoclusters

There are many diverse applications of the nanoclusters of the present invention, including for example, biosensors and catalyst dispensers.

### Biosensors

In one embodiment, the nanoclusters are dimers which can be used as biosensors. The dimer can be used for a method of detecting the presence of a particular target biomolecule in a sample.

A detection dimer comprises a first recognition nano-component attached to a second recognition nano-component. The first recognition nano-component comprises a first nanoparticle and a first specifically-bindable biomolecule. The second recognition nano-component comprises a second nanoparticle and a second specifically-bindable biomolecule. The first recognition nano-component can be attached to the second recognition nano-component directly or by means of a linker. In particular, the first recognition nano-component is attached to the second recognition nano-component by binding of the first biomolecule to the second biomolecule with an initial binding strength. Alternatively, the first recognition nano-component is attached to the second recognition nano-component by a linker which binds the first biomolecule to the second biomolecule. The linker binds the first biomolecule to the second biomolecule with an initial binding strength.

A sample is contacted with the detection dimer. If a target biomolecule is present in the sample, then (i) the target biomolecule binds to either the first biomolecule or the second biomolecule with a detection binding strength; or (ii) the target biomolecule binds to the linker with a detection binding strength. The detection binding strength is greater than the initial binding strength.

In some embodiments, the detection dimer comprises a specifically bindable nanoparticle 1 directly bond to an affinity tag recognition region of linker peptide. The second recognition region of the linker peptide recognizes and specifically binds a biomolecule attached to nanoparticle 2 of the dimer with an initial binding strength. Thus, the second recognition region has low strength affinity to a molecule conjugated to the surface of nanoparticle 2, and a higher affinity to a target biomolecule. The presence of the target biomolecule results in its binding to the affinity tag and the concomitant dissociation of the dimer.

A sample is contacted with the detection dimer. If a target biomolecule is present in the sample, then the second recognition region of the linker peptide binds to the target biomolecule with a detection binding strength. The detection binding strength is greater than the initial binding strength.

An example of a biosensor dimer follows: A linker peptide consisting essentially of affinity tag 1 which directly binds to the surface of a quantum dot, a flexible spacer sequence, followed by the CAR protein. For instance, affinity tag 1 can have the following sequence: NNPMHQN, and bind directly to a ZnS-capped cadmium selenide (CdSe) quantum dot, while the CAR protein moiety would recognize a gold nanoparticle that was functionalized with a modified Knob protein (Knob-M). The modification of the Knob protein creates a weaker affinity between CAR and Knob-M than between CAR and the wild type Knob protein (Knob-wt). Thus the dimer is a quantum dot bound via a protein linker to a gold nanoparticle. The presence of the gold particle would result in quenching of fluorescence of the quantum dot. If the Knob-wt protein is present in a sample, then the CAR protein would dissociate from Knob-M and bind to Knob-wt. This would result in dissociation of the particle dimer, which can be detected due to increased fluorescence of the sample.

It is then determined whether the first recognition nano-component became detached from the second recognition nano-component to form monomers. If monomers were formed to a sufficient level, then the target biomolecule is deemed to be present in the sample.

The nanoclusters of the present invention can also be in the form of trimers which can be used a biosensors. The trimer can be used for a method of detecting the presence of a first target biomolecule and/or a second target biomolecule in a sample.

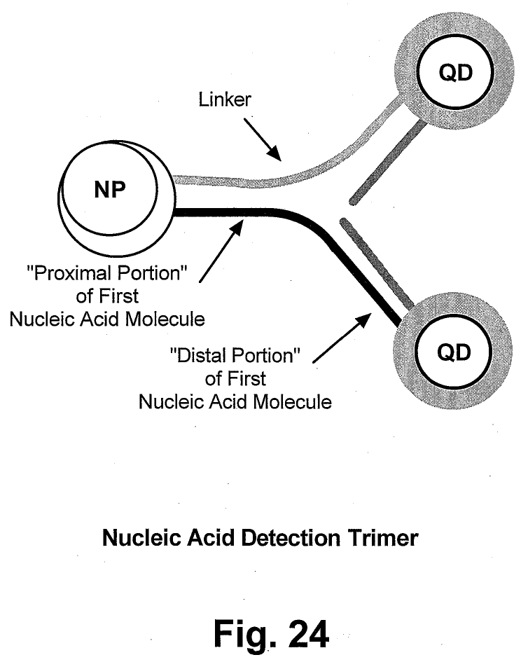

In one embodiment, the detection trimer comprises nucleic acid molecules and is Y-shaped. In this embodiment, the detection trimer comprises a first recognition nano-component attached to a second recognition nano-component and a third recognition nano-component. The first recognition nano-component comprises a first nanoparticle and a first single strand nucleic acid molecule. The second recognition nano-component comprises a second nanoparticle and a second single strand nucleic acid molecule. The third recognition nano-component comprises a third nanoparticle and a third single strand nucleic acid molecule. See FIG. 24. Preferably, the second and third nanoparticles are quantum dots.

The first recognition nano-component is attached to the second recognition nano-component by a single stranded nucleic acid linker which binds two areas. In particular, the linker binds i) the portion of the first nucleic acid molecule which is more proximal to the first nanoparticle to ii) the second nucleic acid molecule. The linker binds the first nucleic acid molecule and the second nucleic molecule with an initial binding strength. The first recognition nano-component is attached to the third recognition nano-component by the binding of i) the portion of the first nucleic acid molecule which is more distal to the first nanoparticle to ii) the third nucleic acid molecule. The first nucleic acid molecule and the third nucleic molecule bind with an initial prime binding strength. The linker preferably contains a spacer sequence which would not specifically bind to the first, second or third single strand nucleic acid molecule.

A sample is contacted with the detection trimer. If the first target biomolecule is present in the sample, the first target biomolecule binds either the portion of the first nucleic acid molecule which is more proximal to the first nanoparticle or binds the second nucleic acid molecule or binds the linker with a detection binding strength. If the second target biomolecule is present in the sample, the second target biomolecule binds either the portion of the first nucleic acid molecule which is more distal to the first nanoparticle or the third nucleic acid molecule with a detection prime binding strength. The detection prime binding strength is greater than the initial prime binding strength.

It is then determined whether the first recognition nano-component became detached from the second recognition nano-component and/or third recognition nano-component to form dimers and/or monomers. If dimers and/or monomers were formed to a sufficient level, then the first target biomolecule and/or second target biomolecule is present.

Trimers can also be formed with biomolecules other than nucleic acids. Typically, these trimers have a fairly linear structure. An example of such a biosensor trimer follows: an affinity tag with low strength binding to a molecule conjugated to the surface of particle 1, a spacer sequence rich in Ser and Gly, a region with one or several cysteines or histidines that allow binding to the surface of the second particle, a linker sequence rich in Ser and Gly, and an affinity tag with low strength binding to a molecule conjugated to the surface of particle 3; preferably, particles 1 and 3 are quantum dots with different fluorescent properties, and particle 2 is a gold particle. Depending on the surface modification of the gold particle, thiol chemistry can be used to bind the cysteines, or Ni-NTA modification will allow binding of histidines.

There are numerous methods by which to determine whether the dimers and trimers of the present invention disassemble to form monomers and/or dimers.

In one embodiment, dimer/trimer disassembly is determined by Dynamic Light Scattering. In particular, the hydrodynamic diameter of a molecule in solution is determined from a measurement of the mobility (in terms of the translational diffusion coefficient in dilute solution) of the molecule by dynamic light scattering experiments.

In another embodiment, dimer/trimer disassembly is determined by fluorescence. In particular, in this embodiment, a recognition nano-component has fluorescent properties (e.g., a quantum dot) that are quenched by the close proximity and/or attachment of another recognition nano-component (e.g. a gold particle).

In order for a metal nanoparticle to quench the fluorescence of a quantum dot, there must be a maximal distance between the quantum dot and the metal nanoparticle, as known in the art. The length of the spacer sequences in a dimer or trimer can be adjusted to create the optimal length with maximum quenching.

Dimer disassembly will result in fluorescence. Depending on the equipment utilized, fluorescence can be detected at many different levels, including minute levels; For example, the fluorescence of a single quantum dot can be measured, thus detecting the presence of a single target biomolecule.

Other methods by which to detect dimer/trimer disassembly include plasmon shift (spectroscopy) and gel electrophoresis.

The biosensors can be constructed to detect any type of target biomolecules by methods known in the art. Clinical applications or applications related to Homeland Security may require detection of any organism of interest. For example, not only human viruses (such as, for example, human immunodeficiency virus (HIV), hepatitis C virus (HCV), hepatitis B virus (HVB), small pox, Ebola, human papilloma virus (HPV), and flu viruses) but also viruses of agricultural importance, (such as, for example, viruses causing hoof and mouth disease), bacteria (including, for example, Anthrax, Staphylococcus, Streptococcus, and Borrelia), and infection-causing protozoa.

Also, the method can be used for the real time detection of toxicity markers, including the detection of specific stress proteins, and stress induced changes in expression and modification of DNA and RNA. In particular, the methods could be used to detect changes in gene expression, changes in gene regulation detectable via the induction of specific siRNAs, SNP, target DNAs secreted by specific tumor lines, etc.

### Binding Strength

### Binding Strength Between Nucleic Acids

The binding strength between two single-stranded nucleic acids is based on the strength of hybridization between the nucleic acids.

The strength of hybridization between two single-stranded nucleic acids can be adjusted by routine experimentation to achieve proper functioning. For example, the strength is a function of the length of hybridized nucleotides, the G-C content, and number of destabilizing mismatches.

In particular, the greater the length of hybridized nucleotides between two nucleic acid molecules, the greater the binding strength is between the two nucleic acids. In one embodiment, the first biomolecule and the second biomolecule of a detection dimer has about 10% to about 30% less hybridized nucleic acids between them as compared with the hybridized nucleic acids between a target biomolecule and the first biomolecule or the second biomolecule. For example, the first biomolecule and the second biomolecule of a detection dimer can have about 8 hybridized nucleic acids whereas the target biomolecule and the first biomolecule can have about 10 hybridized nucleic acids.

In another embodiment, if the releasing biomolecule, the anchoring biomolecule and the biomolecule on the initial recognition-nano-component (hereinafter “initial biomolecule”) are nucleic acids, then the length of the hybridized nucleotides in the releasing molecule and the anchoring biomolecule is greater than the length of the hybridized nucleotides in the anchoring molecule and the initial biomolecule. In addition to length, the strength of the hybridization can be reduced between the initial biomolecule and the anchoring biomolecule vis-a-vis the releasing biomolecule and the anchoring biomolecule by decreasing the G-C content and/or by inserting destabilizing mismatches in the nucleotides.

### Binding Strength Between Biomolecules Other Than Nucleic Acids

For the detection of biomolecules other than nucleic acids, the principle is based on the initial binding strength between two biomolecules with low affinity (but strong enough to keep the dimer pair stable). Examples of such biomolecules include proteins, peptides, cytokines, and specific metabolites. Subsequent binding of a biomolecule showing superior affinity to one of the binding partners, or the linker, results in dimer dissociation.

An example follows. A His-tagged CAR (coxsackie and adenovirus receptor) protein functionalizes particle 1, and has low affinity binding to particle 2 (e.g., a quantum dot) functionalized with a modified Knob protein. The modification of the Knob protein is a change in an amino acid in the epitope region. Such modification provides the low affinity binding. Once the wild-type Knob protein is available, e.g. because the adenovirus is present in a sample, the CAR protein and wild-type Knob protein will bind with high affinity, resulting in dimer dissociation. Alternatively, an affinity tag can be used (instead of the CAR protein) which recognizes the Knob protein. The affinity tag shows a strong binding to the wild-type protein, and a modified Knob protein (modified in the epitope region recognized by the affinity tag) is used to functionalize the second particle in the binding pair.

This method can revolutionize the field of real time detection, including that of toxicity markers. For instance, the method can be used to detect a specific antigen (to which the antibody used in the dimer formation has a superior affinity), but also for antibody detection when binding of a low affinity antibody is replaced by that of a high affinity antibody.

### “Strawberry Structures.”

“Strawberry structures” were discussed above. A strawberry nanocluster detector comprises a metal nanoparticle and a plurality of attached fluorescent quantum dots. The metal nanoparticle is the “main” nanoparticle and the quantum dots are the “small” particles. For example, about five quantum dots can be attached to the metal nanoparticle. The quantum dots are attached to the metal nanoparticle by specifically bindable biomolecules. The quantum dots fluoresce at different colors. The quantum dots are quenched when attached to the metal nanoparticle. The specifically bindable biomolecules which attach the quantum dots to the main nanoparticle are different from one another and correspond to a particular color quantum dot. Thus, the strawberry nanocluster can detect as many target biomolecules as it has quantum dots.

Upon exposure of the strawberry nanocluster detector to a sample, a particular quantum dots will be released if a specific target molecule binds to the bindable biomolecule with higher affinity than the quantum dot binds to the main nanoparticle. When the quantum dot detaches from the main nanoparticle, it fluoresces at a particular color thereby indicating the presence of a particular target biomolecule in a sample.

In another embodiment, the “small” nanoparticles of a “strawberry-structure” each contain different catalyst thereby forming a multifunctional catalyst system.

### EXAMPLES

### Example 1

A demonstration is given of how 10-nm nanoparticles can be functionalized with anisotropic binding interactions using a solid-state assembly approach. This approach utilizes DNA-derived interactions at nanoscale interfaces in a step-by-step approach. The approach is highly modular, and produces high quality and high yields of building blocks in a first of its kind high throughput fashion.

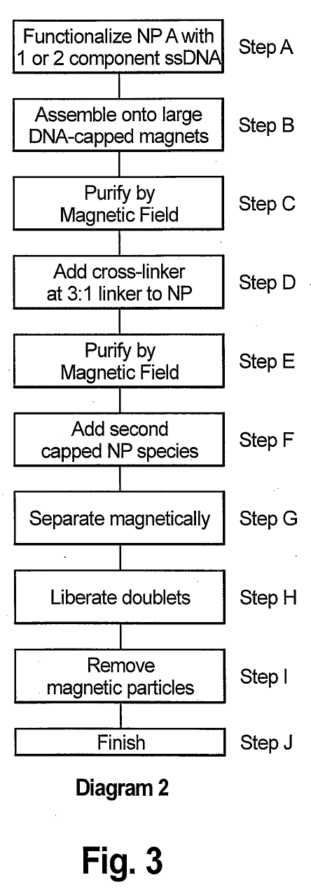

A simplistic view of the novel process is depicted in FIG. 3, Diagram 2. A first group of nanoparticles is functionalized with one- or two-component ssDNA (Step A). These functionalized nanoparticles are assembled onto large (micrometer sized) DNA-capped magnetic particles (Step B). The solution is purified by using a magnetic field (Step C), a process taking a few minutes and introducing no additional contaminants. The typical yield at this stage, and at all succeeding stages, is generally greater than 98%. A cross-linker is added at a surprising ratio of 3:1::cross-linker:nanoparticle (Step D), after which the system is again purified in a magnetic field with only the addition of fresh buffer solution (Step E). A second capped species of nanoparticle is added (Step F) to the solution. It is important to note that no separation based on the number of DNA per nanoparticle has been undertaken. Species are separated magnetically (Step G), after which doublets (dimers) are liberated from the magnetic substrates (Step H). After removal of the magnetic particles (Step I), the processing is complete (Step J) and the resulting solution has a high concentration of dimers with very few assemblies having more than two DNA per nanoparticle.

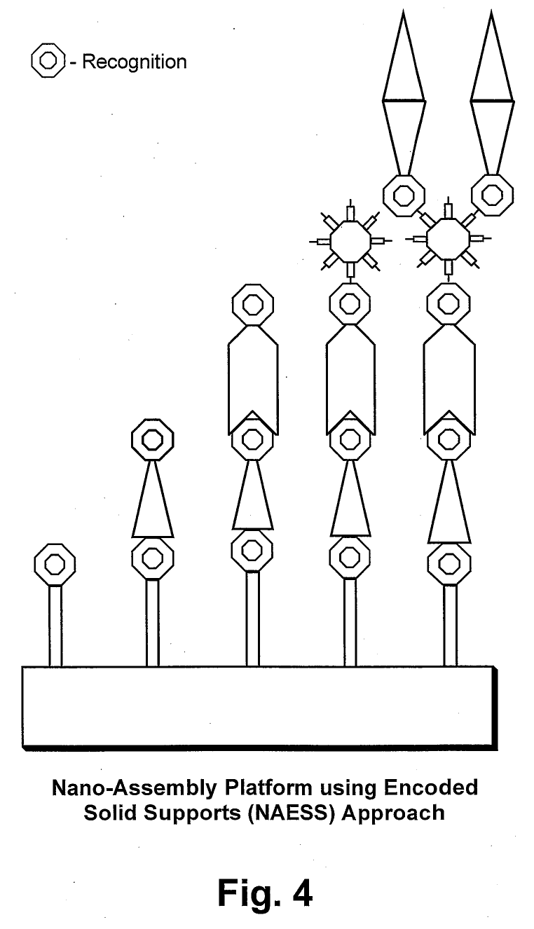

Scheme 2 in FIG. 4 shows a generalized approach towards the controlled assembly of different interacting nano-objects at a colloidal interface. Each step, or layer, depends on a specific recognition site being available for assembly. Harnessing the multiple recognition sites of biomaterials (DNA, proteins, peptides, etc), it is possible to have a virtually unlimited number of encodings.

In a particular example of the application of the inventive technology, DNA-encapsulated nanoparticles and magnetic-colloid supports were utilized (Scheme 3). Each assembly step is encoded for specificity via a particular 15- to 20-base-pair double-stranded DNA interaction. After each step the system is purified via a magnetic field, and clean solution and products are then added, which aids fabrication fidelity. This magnetic purification process takes place in minutes in a benign environment and typically yields ≧98% desired product.

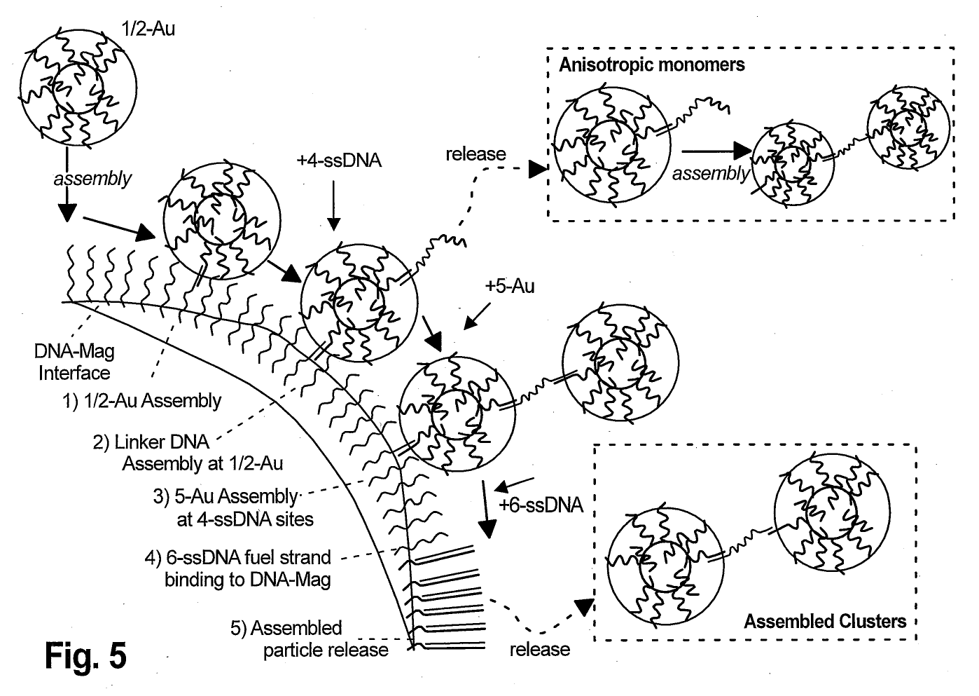

Scheme 3 in FIG. 5 is a schematic illustration of the nano-assembly platform using encoded solid supports (NAESS) approach. 2, 3-capped Au particles are assembled onto a 1-capped magnetic colloid (step-1). After purification of any un-assembled Au, 2-ssDNA cross-linkers are assembled onto layer 1 (step 2). After purification of any un-assembled 4-ssDNA, 5-capped Au particles are added as a second layer and assembly via complementary hybridization of the 4-ssDNA. After purification of any un-assembled 5-Au, a 6-ssDNA fuel strand is added, which binds to the 1-Mag, and liberates the assembled nanostructures. Finally, a 7-ssDNA quenching strand is added, which limits any further cross-linking and preserves the nanostructured dimers.

Thus, a novel nano-assembly platform using encoded solid supports (NAESS) for the fabrication of nanoparticle monomers with controlled anisotropy is demonstrated. Accordingly, well defined doublet groupings, or Janus-type constructions, can be made. Anisotropy is driven by DNA interactions in a layer-by-layer fabrication. Scheme 1 shows an illustration of the NAESS approach. Gold nanoparticles (Au, 11.1±1.2 nm), were first functionalized with either a one- or two-component single-stranded DNA (ssDNA) shell structure following both established protocols and other recent work, and then assembled in a step-wise manner onto large (1-4 μm) DNA-capped colloidal magnetic particles. (NOTE: In the description that follows, elements are mapped to the figures by way of reference numerals which are found before the name of the element.) In step 1, a DNA-capped gold nanoparticle of mixed ssDNA composition (1-ssDNA, 2-ssDNA: [1]=[2]), may be used to facilitate moderate binding of the 2-ssDNA via 15 bp hybridization to a 1-ssDNA capped magnetic colloid (1-Mag), while preserving the 3-ssDNA as a second addressable binding site. After assembly of the 2, 3-Au to the 1-Mag, 1-3 hrs, and subsequent solution color change from ruby-red to clear, the system was purified via magnetic field, and washed via multiple rinsing with PBS buffer (10 mM phosphate buffer, 0.2 M NaCl, pH=7.4). In step 2, a 33-base cross-linker (4-ssDNA) is added at a ˜3× ratio ([4-ssDNA]/[2, 3-Au]=3), and allowed to anneal for 5-12 h under stirring before the supernatant solution is removed, and purified. In step 3, a 5-capped Au, which is complementary to the free linker end of the 4-ssDNA cross-linker, is added at a strict 1:1 ratio with the first particle layer (i.e., [2, 3-Au]/[4-Au]=1) and allowed to react for 3 h. Upon magnetic separation, the solution is optically clear, signaling the absorption of the second Au to layer 1 at the 1-Mag solid-support. Control experiments with non-complementary nanoparticles revealed less than 1% non-specific adsorption over 5 days. After each step, assembly yields were calculated based on UV-visible measurements (UV-vis), and typically yielded >98% of particle assembly to the solid support.

Once purified, an excess (1000×) of a 6-ssDNA fuel strand is added to the solution ([6-ssDNA]/[1-ssDNA]=1000) and is mixed gently for 4-12 h. The fuel 6-ssDNA selectively binds via 18 bp to the 1-ssDNA at the 1-Mag interface, which replaces the 15-bp binding of the 2-ssDNA of the 2, 3-Au interface, thus liberating the assembled doublet without the need to increase temperature (T=25° C.), or perform DNA ligation. The product solution was shown to gradually become red, illustrating the release of gold nanoparticle dimers into solution. Once disassembled, a 15-base, 7-ssDNA ([7-ssDNA]/[4-ssDNA]=1), is added to the solution, which effectively passivates any free cross-linkers in the system (e.g., 4-ssDNA), thus limiting any further assembly after release solution. The products could then be cleansed free of any 1-Mag via magnetic field, and additionally purified via a 0.2 μm sephadex filter. The assembled Au dimers were then stored at 4° C. in PBS buffer.

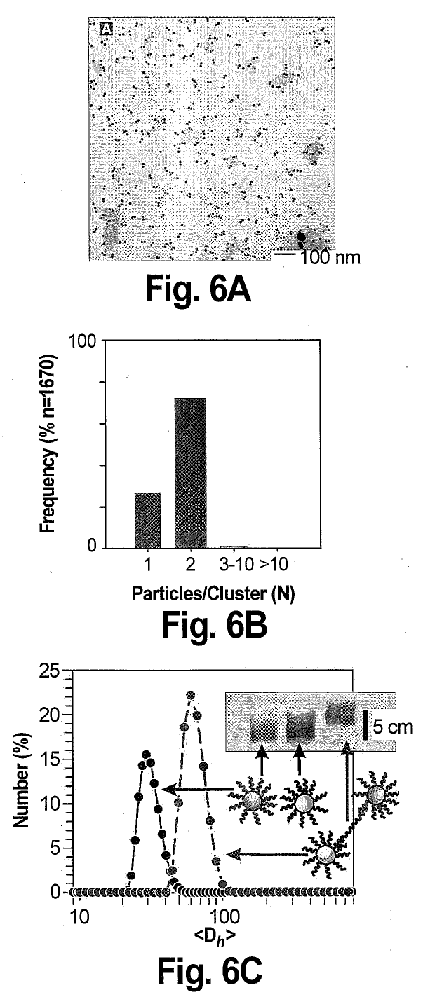

FIG. 6a shows a typical TEM micrograph of the released NAESS products. Clearly, the particles assembled into the pre-designed, doublet morphologies, as was demonstrated with multiple grid locations, samples, and trials. Statistical analysis of multiple TEM micrographs demonstrate a remarkable assembly yield of 70˜85% doublet morphologies (FIG. 6b), with 15˜30% un-assembled 2, 3-Au, and only ˜1% of aggregates with three or more particles. In addition to these high yields, and lack of large aggregates, the micrographs also demonstrate the ability of this approach to produce relatively high concentrations of doublets ([Au]>100 nM, 100-2000 μl). It is also important to note that these micrographs are collected for samples without additional purification, such as gel electrophoresis or HPLC, after release from the 1-Mag supports, which illustrates the high throughput fashion of NAESS fabrication. These assembled doublets were also found to be extremely robust, as illustrated by imaging a 15-, 30-, and 65-day aged solution, which revealed similar morphology and doublet yields. The dimer morphologies could also be observed using agarose gel electrophoresis and dynamic light scattering (DLS). FIG. 6c shows the decreased electrophoretic mobility of the dimers compared to monomers, while DLS results revealed an increased hydrodynamic diameter (Dₕ) of 80˜100 nm for the assembled doublets, compared to the isolated 2, 3-Au signature of Dₕ˜21 nm.

The observed dimer construction and release from a solid support via entirely DNA-based interactions is remarkable given no change in assembly conditions (i.e., temperature, ionic strength, etc.) is required. However high dimer yields were not attained for all assembly conditions used. First, 2,3-Au loading was used on the 1-Mag of only ˜50% of the accessible DNA at the 1-Mag, thereby limiting the ability of the second layer of Au (i.e., 5-Au) to bridge Au of the first layer, a result which was observed in early experiments at maximum adsorption. Secondly, a ssDNA cross-linker ratio of ˜3× ([2, 3-Au]/[4-ssDNA]=3) dramatically increased the assembly kinetics and yield of the doublets, compared to an obvious choice of a 1× ratio. This may be due to standard errors in DNA or Au concentrations, or to the possibility of single Au particles receiving multiple cross-links at a ˜1× ratio (i.e., stochastic distribution). The use of the 7-ssDNA quenching strand was observed to greatly enhance the stability of the released doublets, limiting additional cross-linking of any free 4-ssDNA cross-linkers. Ease in purification of the system after each assembly step via magnetic separation greatly enhanced assembly yields, removing excess nanoparticles and DNA cross-links, thus limiting any re-assembly in the solution.

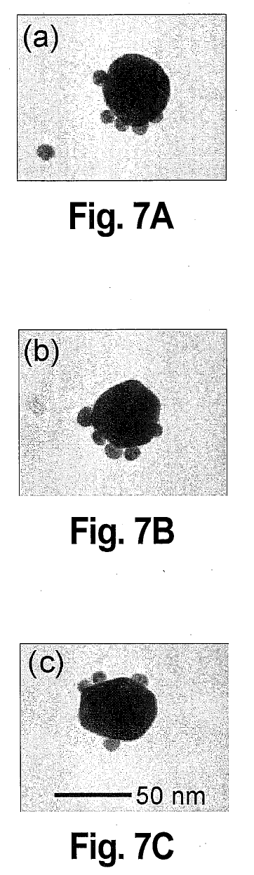

In addition to this step-by-step assembly of highly discrete nanoparticle clusters, the NAESS approach can be utilized to fabricate nanoparticles with a high degree of anisotropic binding character. Such anisotropy is extremely limited in particle nanosystems due to the complexities of using purely surface chemistry based particle modification. However, examples of anisotropic bindings, including colloidal particle systems, have been observed. In addition, initial results using nanoparticles of different sizes have been reported by Mirkin and co-workers. FIG. 7 illustrates the use of NAESS for anisotropic monomer fabrications. In this system, a large 2, 3-capped gold nanoparticle (˜50 nm) is assembled onto the 1-Mag via step 1, as previously illustrated (FIG. 6b). The immobilized particle is then hybridized with the identical 4-ssDNA linker strand at ˜5× ratios. Upon linker hybridization for ˜6 hr, the system is purified, and the particle is released from the 1-Mag via the addition of 6-ssDNA, as described above. The released particles, with anisotropic binding character (via added cross-links), are then combined with a ˜5× molar ratio of smaller 5-Au (˜11 nm), and allowed to assemble overnight in solution. The assembly products, as characterized with TEM, are shown in FIG. 7. The final morphologies revealed a high degree of anisotropic assembly cluster, with ˜5 small Au (5-Au) per large particle. The most striking feature of these clusters is the hemispherical binding of five small particles positioned/assembled at the large particle, with >80% of samples revealing these Janus-like morphologies.

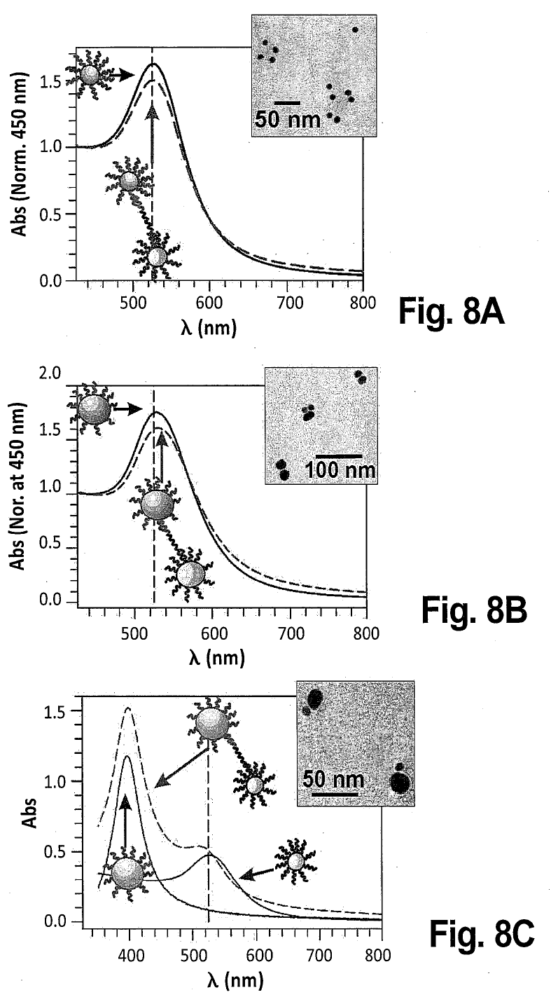

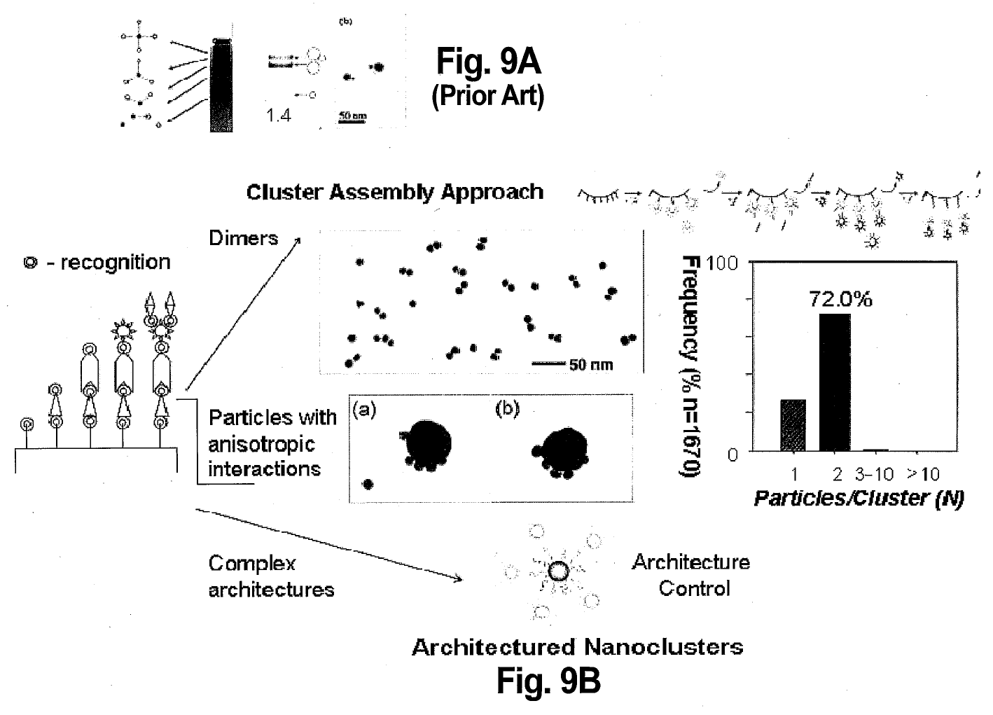

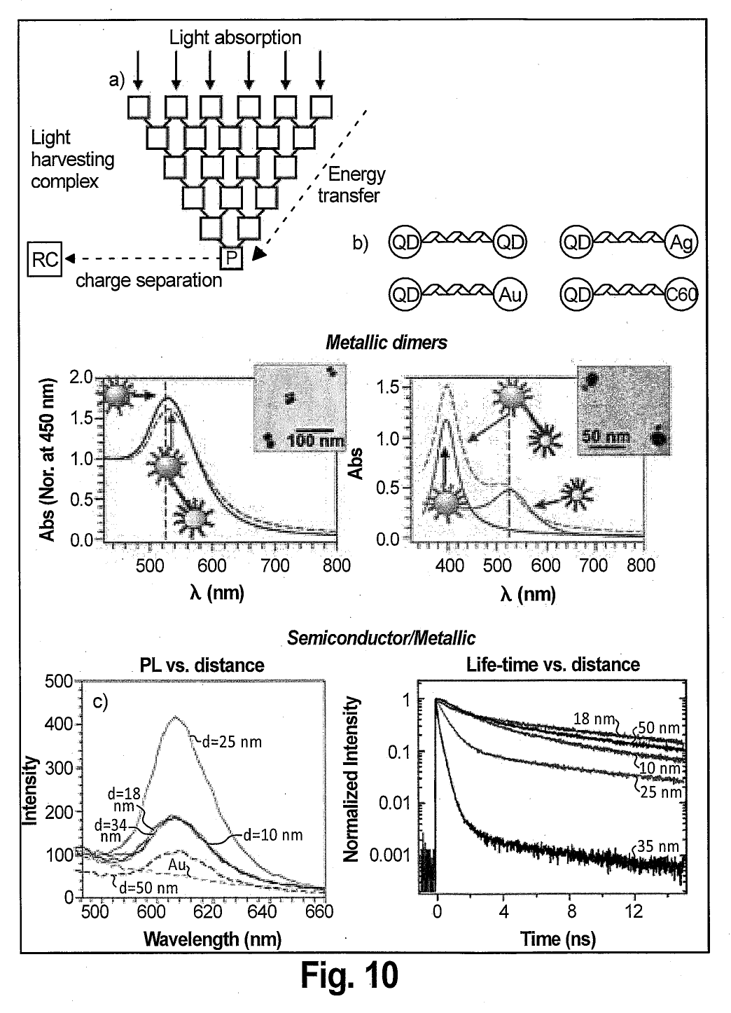

Great numbers of researchers focus on the optical, scattering, and hydrodynamic properties of these types of assembled materials, including clusters and particularly dimers. To gain insights into the characteristics of the described systems, we employed UV-visible spectrophotometry (UV-vis), dynamic light scattering (DLS), and in situ small angle x-ray scattering (SAXS) measurements (FIG. 8). UV-vis is employed to probe the surface plasmon (SP) resonance band of Au, which is associated with isolated Au and assembled nanostructures. In assembled systems, a red shift (a shift to longer wavelengths, i.e. lower energy) in the SP-band peak position is indicative of a decrease in interparticle distances and a possible increase of aggregate size. DLS meanwhile probes the hydrodynamic diameter (Dₕ) of the ssDNA-capped Au and their assemblies, which is related to aggregate size, interparticle interactions, and cluster geometry. SAXS meanwhile provides insights into the spatial microstructure of assembled nanosystems under in situ conditions. FIG. 8a shows the UV-vis spectra for the isolated 1, 2-Au, and assembled doublet systems along with a TEM micrograph sampled from the studied solution. We observed that the SP-band of the isolated Au and assembled doublets are very similar in both SP-band position and profile, with only a consistent decrease in extinction being observed. This finding is correlated with recent work on modeling SP-band characteristics via Mie theory and modified Mie theory, which have illustrated that indeed, only a small SP-band red-shift is expected for nanostructures whose interparticle distances (d) are greater than ˜½ the nanoparticle diameter (D). In this system, D˜11 nm is indeed comparable to interparticle distances measured via in situ SAXS, which revealed a surface to surface interparticle distance of d˜9.5 nm, for the assembled doublets. Both the SP-band and SAXS measurements appear influenced by the presence of a small quantity of isolated Au monomers.

An additional strength of the NAESS fabrication method is its modular design, which allows for the use of different cross-linking ssDNA, e.g., for length control, as well as multiple sized or multi-component nanoparticles, such as hetero-dimers. FIG. 8 illustrates this ability, revealing the SP-bands and TEM micrographs for Au—Au dimers with larger particles, D˜23 nm (B), as well as heterodimers of Ag—Au, (D_{Ag}˜30 nm, D_{Au}˜11 nm) (C). For larger Au—Au doublets (D_{Au}˜23 nm), which uses the same ssDNA of the previous samples, an increase red shift of the SP-band of ˜8 nm is observed, accompanied by decreased extinction and slight broadening. Interestingly, an assembled Ag—Au doublet revealed characteristics of both the Ag SP-band (˜410 nm) and the Au SP-band (525 nm), with the Ag SP-band showing a 5 nm red-shift, and the Au SP-band showing a blue-shift of ˜5 nm, a shift that was not observed in a physical mixture of particles. These results indicate that the assembled nanoparticle doublets, with well defined interparticle distances and interactions, can serve as a platform for studies focused on understanding interparticle distance (d) influence over SP-band properties and electromagnetic coupling phenomena, such as the use of these assembled dimers for model studies on metal enhanced fluorescence (MEF) processes. For example the charge transfer between metallic nanoparticles and the emission properties of quantum dots (q-dots) may be measured via assembled heterodimers of Au—CdSe/ZnS (D_{Au}˜11 nm, D_{q-dot}˜5 nm, d˜11 nm) which reveal strongly quenched photoluminescence properties, in agreement with recent theoretical modeling of SP-band to q-dot energy transfer with small metallic particles, which is part of ongoing research efforts.

### Example 2

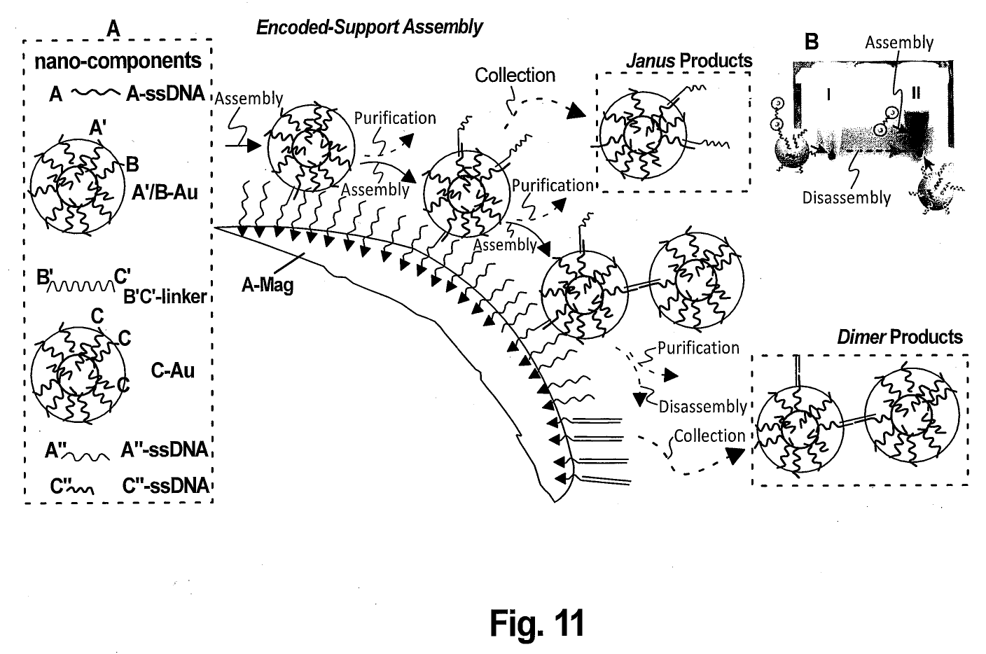

A schematic of the developed approach, and the required nano-components (particles, DNA, etc) are shown in FIG. 11, in-which the fabrication of nanoparticle clusters, such as dimers, or Janus particles, is illustrated. In a typical experiment, gold nanoparticles (Au, 11.1±1.2 nm) are first functionalized with either a single-, or two-component single stranded DNA (ssDNA) shell structure, (21, 22) then assembled in a step-wise manner onto large (1-4 μm) A-capped magnetic colloids. (A-Mag, Table S1). For instance, the A′/B—Au, ([A′]=[B]) was used to facilitate moderate binding to A-Mag, while preserving the B-ssDNA as a second addressable binding site. (22) Upon assembly to the A-Mag (via A to A′ dsDNA duplex formation), accompanied by a calorimetric change from ruby-red to transparent, the system was purified by magnetic field, and rinsed multiple times with buffer. Next, the 33-base B′C′-ssDNA linker is added to the A′/B—Au layer (step-2), and allowed to anneal before the system is again purified. In step-3, C—Au is added, which hybridizes to the complementary C′ fragment of the B′C′-ssDNA linker. Upon hybridization, the solution is again optically clear, signaling the assembly of the second C—Au layer. Control experiments with non-complementary DNA-capped Au revealed less than 1% non-specific adsorption of C—Au to the A-Mag interface over 12 h. Finally, an excess of a A″-ssDNA “fuel” strand (23) is added to the A-Mag containing two layers of Au. The A″-ssDNA selectively binds via a 23-bp duplex formation with A-ssDNA on A-Mag, replacing the 15-bp A′/B—Au linkage, thus liberating the assembled structure. The use of fuel strands allowed for the entire assembly system to proceed without requirements of increased temperature, or DNA ligation, as was shown by Mirkin and co-workers to work effectively in separating particles from colloids.(24) In addition, a 15-base C″-ssDNA was added to quench any free B′C′-ssDNA linkers in the system, suppressing any further aggregation after release.

FIG. 11(A) shows an idealized illustration of the assembly and release of nanoparticle dimers or Janus particle monomers at an encoded support. Briefly, A′/B—Au particles are assembled onto the surface of A-ssDNA grafted magnetic colloid (A-Mag, step-1), via A- to A′-ssDNA hybridization. Upon magnetic purification, a layer of B′C′-linker is added, which hybridizes via B′ to B-ssDNA on A′/B—Au (step-2). Due to the geometrical constraints and steric repulsion between non-complementary ssDNA, the hybridization of B′C′-linker only occurs on the top hemisphere of the particle, which results in anisotropically functionalized particles. After purification, a second particle layer of C—Au is added via C— to C′-ssDNA hybridization (step-3). Finally, the A″-ssDNA fuel strand is added, which preferentially binds to A-Mag, and liberates the assembled clusters (step-4a) or anisotropic monomers (step-4b). During this process, the C″-ssDNA is added, which limits any further cross-linking by quenching the C′ sites of B′C′-linker, thus preserving the nanostructured dimers (step-5). FIG. 11(B) An optical micrograph of the assembly system under magnetic separation; for samples with dimers bound to A-Mag (I), and after release into solution (II).

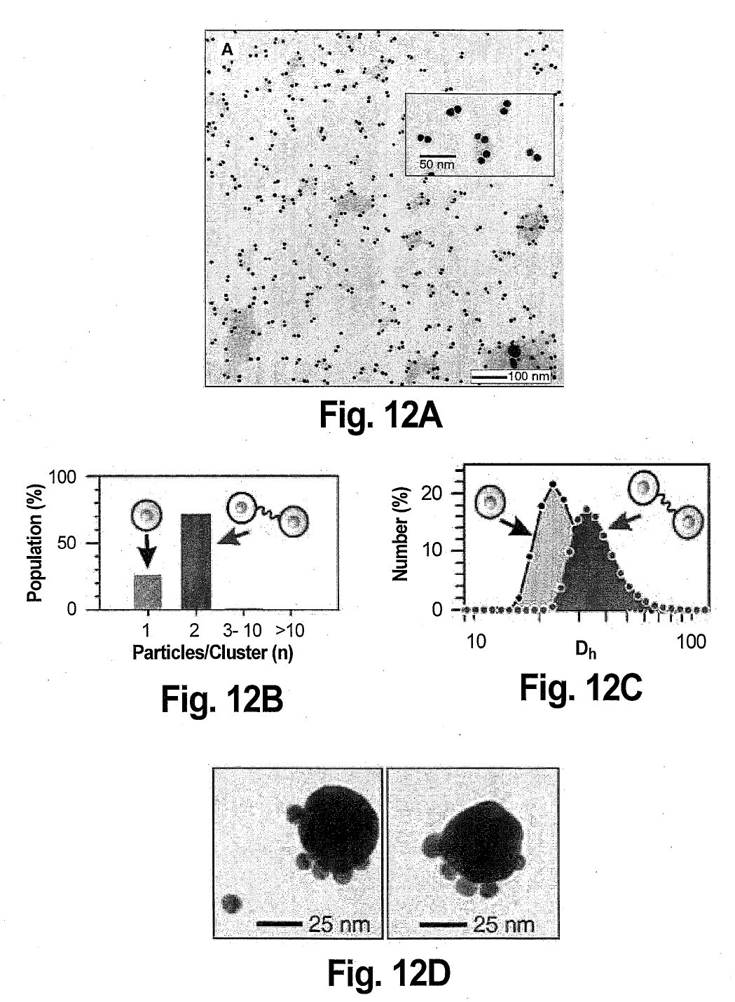

These assembled and released nano clusters were collected and visualized using Transmission Electron Microscopy (TEM) without any additional purification procedures. FIG. 12A reveals a typical TEM micrograph demonstrating the remarkably successful fabrication of structures containing two nanoparticles, i.e. dimers. A representative statistical analysis (FIG. 12B) reveals a ˜73% dimer yield, along with ˜26% monomers, and only ˜1% clusters containing more than 2 particles. In addition to these high product yields, and lack of larger aggregates, the micrographs also demonstrate the ability of this approach to produce high concentrations of dimer products, scalable from nano- to micro-molar concentrations, which is challenging to achieve using existing methods.

FIG. 12(A) shows a representative TEM micrograph for nanoparticle dimers assembled and released via the encoded solid-support approach, sampled without additional purification or size selection. FIG. 12(B) shows a statistical analysis of the TEM micrograph, revealing ˜73% dimer morphology, ˜26 % monomers, and only 1% structures with 3 or more particles (n=1670 particles). (C) The DLS measured Dₕ population for C—Au monomers, and assembled dimers. (D) A representative set of TEM micrographs sampled after the assembly of 50 nm A′/B—Au Janus particles with a ˜5× ratio of 11 nm C—Au.

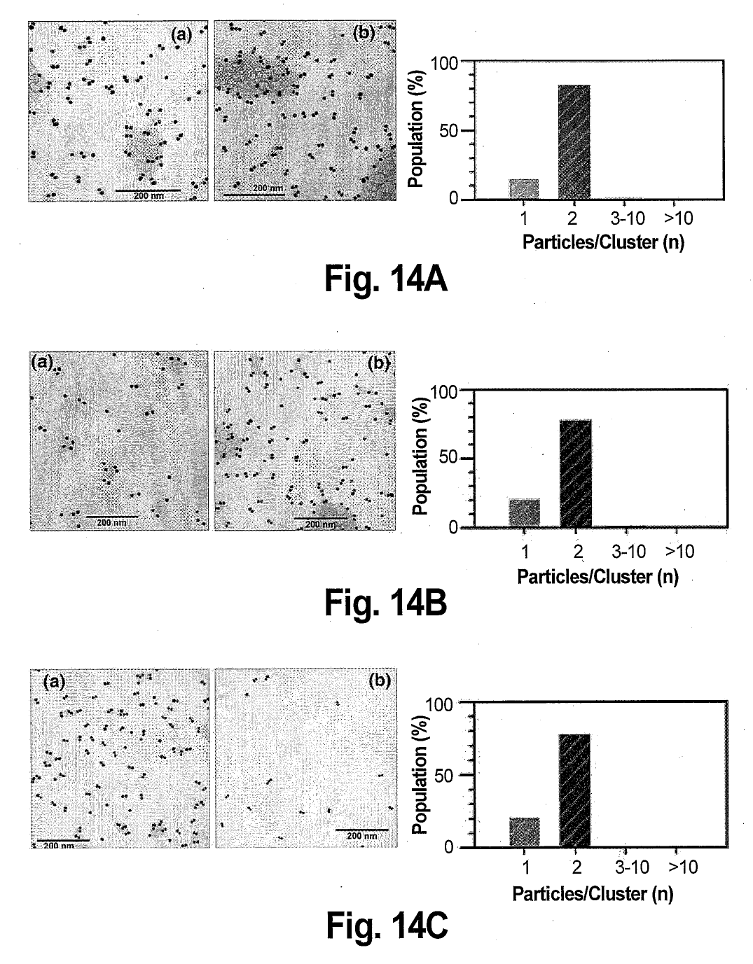

Similar results were visualized at multiple TEM grid locations, and reproducible trials with dimer yields consistently between 70-83% (FIG. 14).

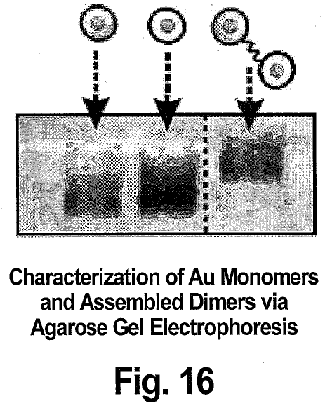

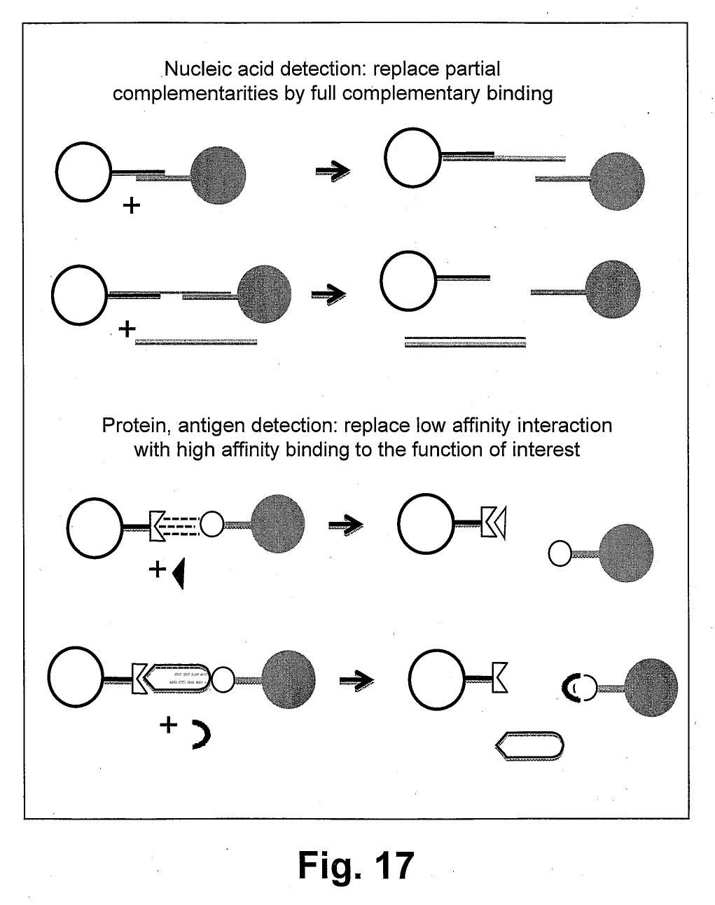

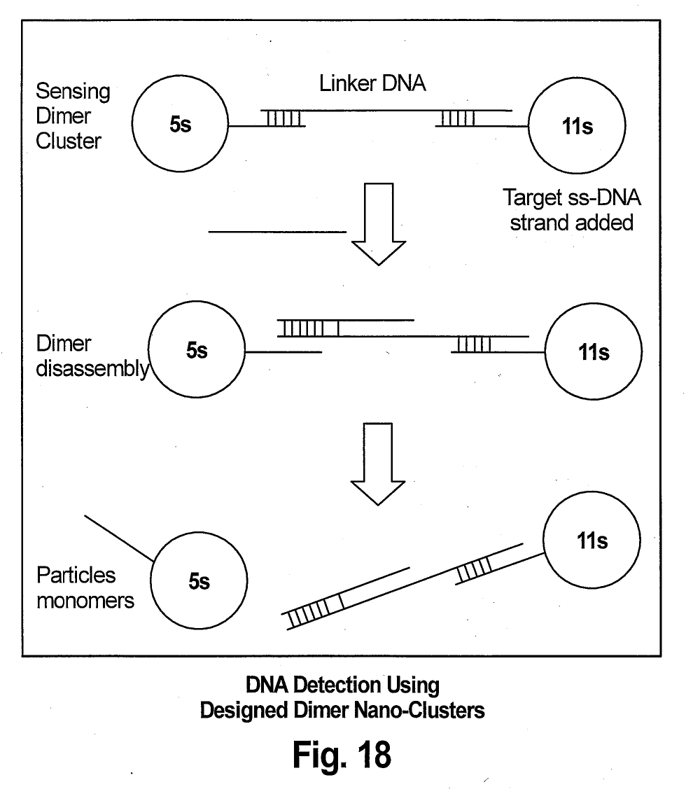

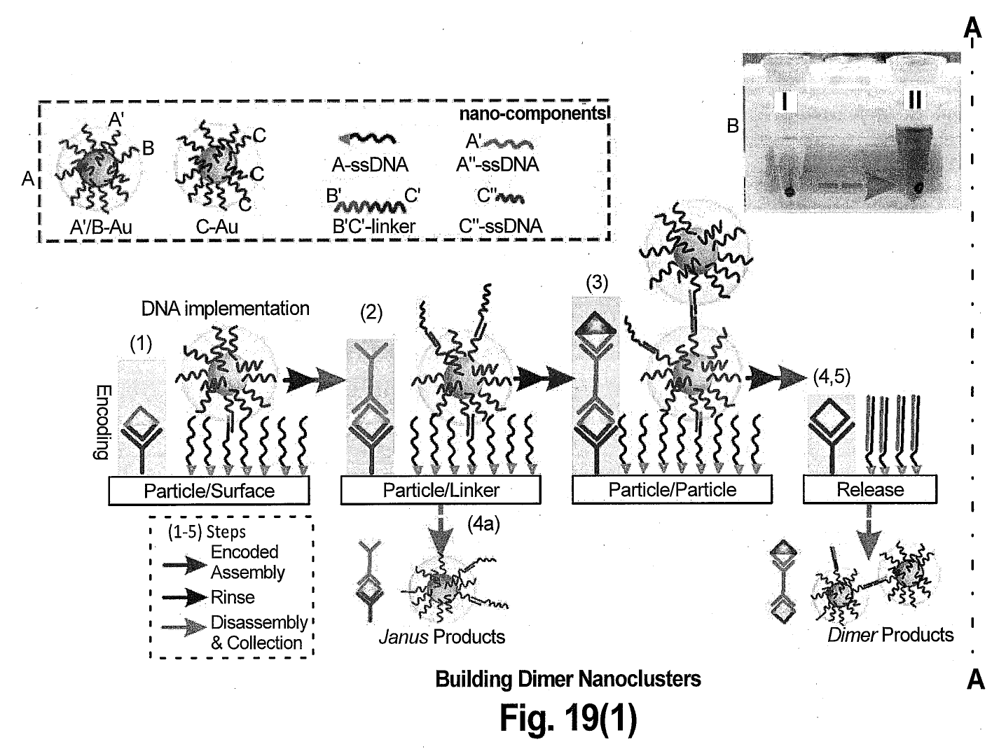

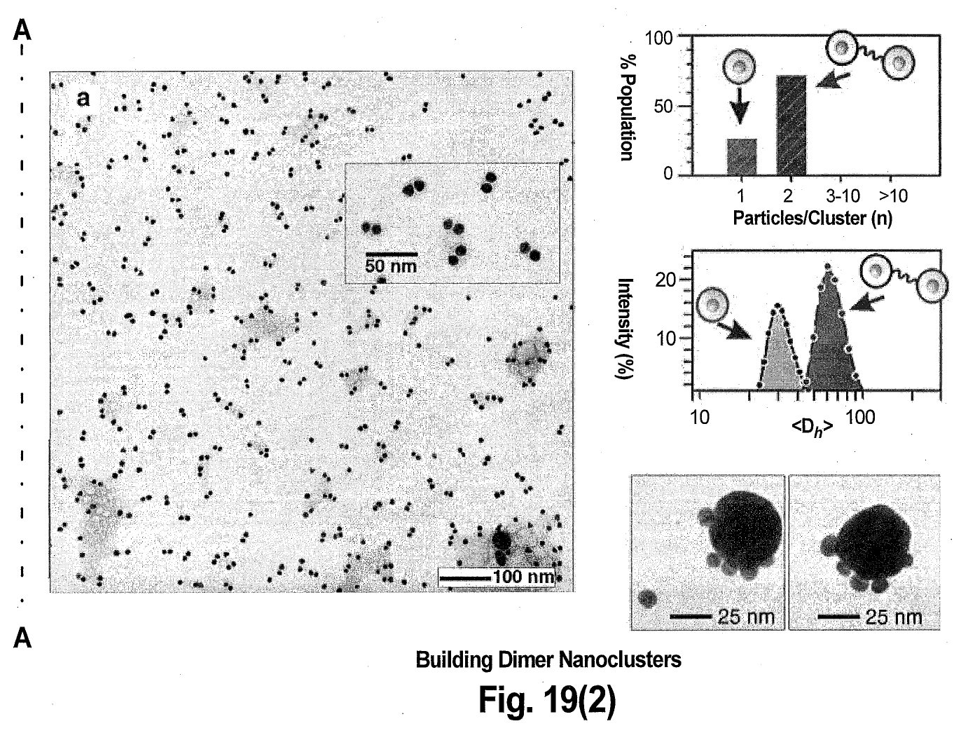

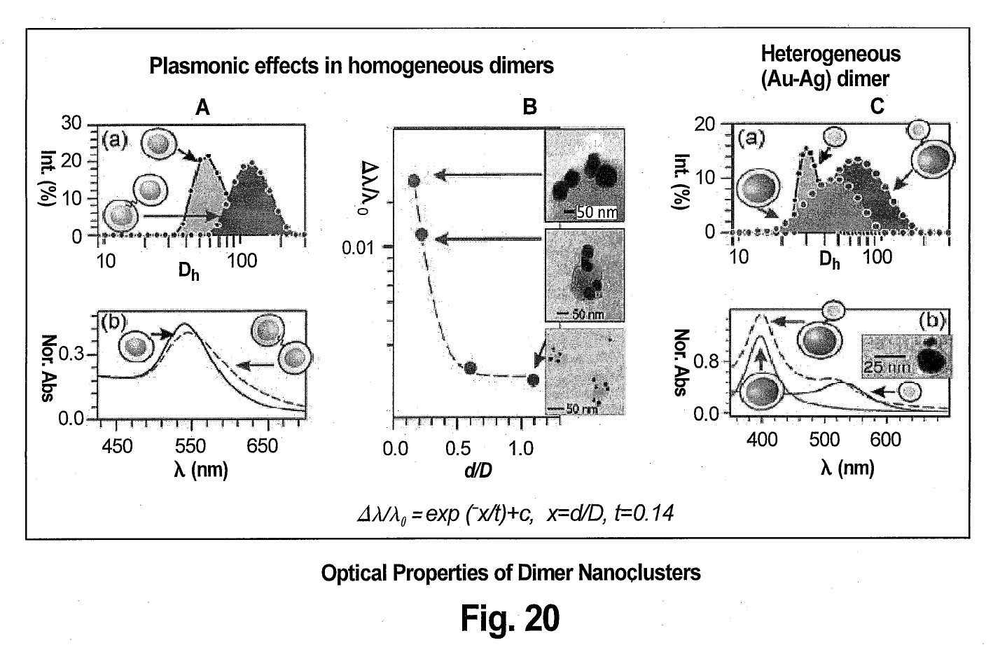

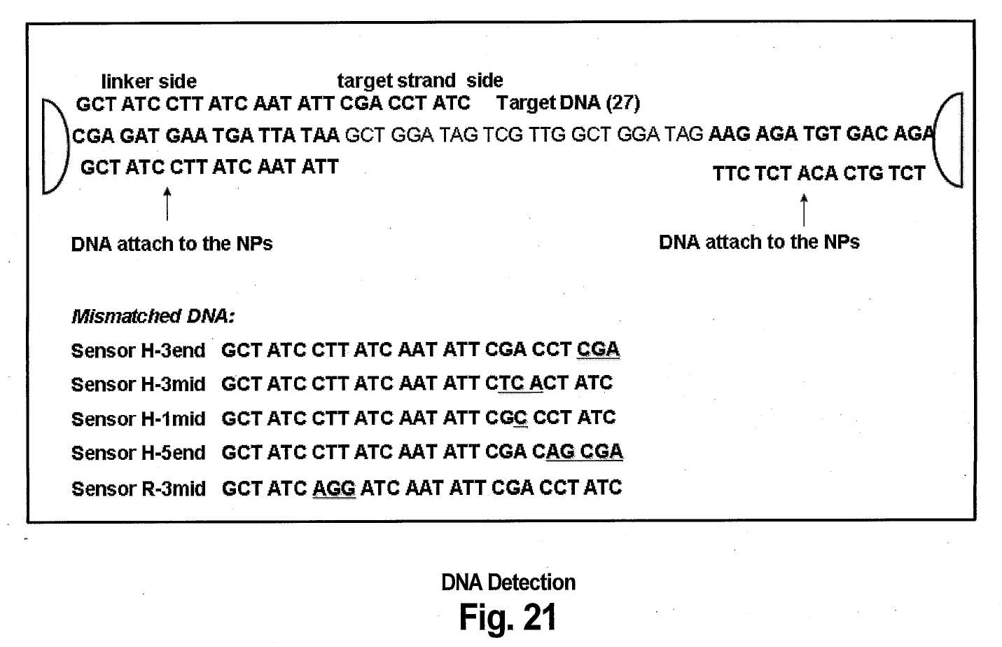

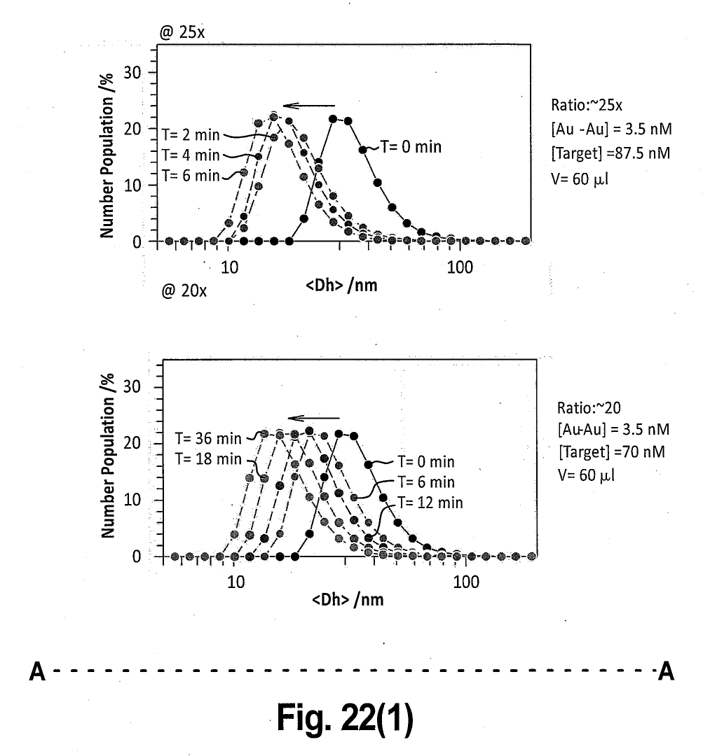

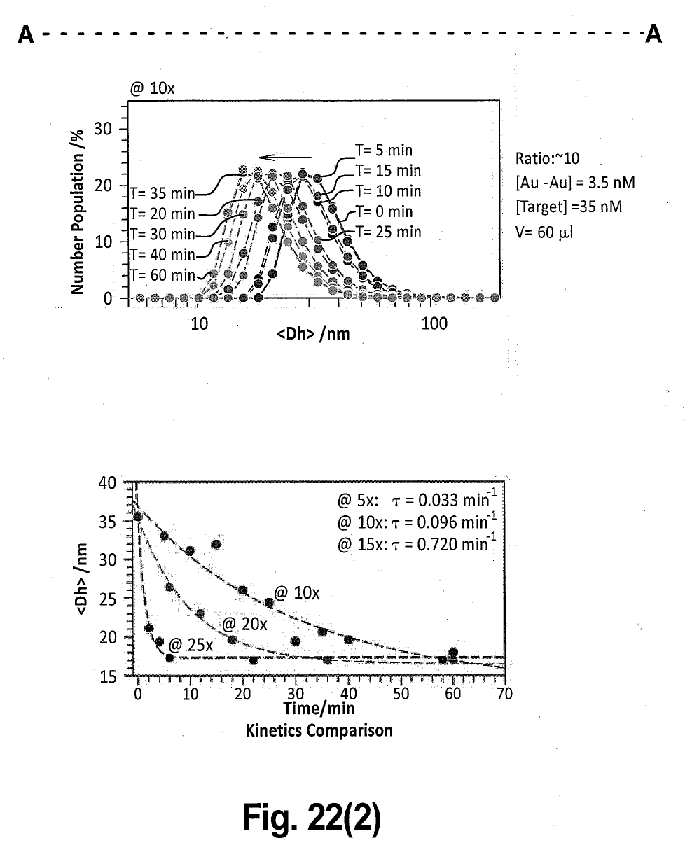

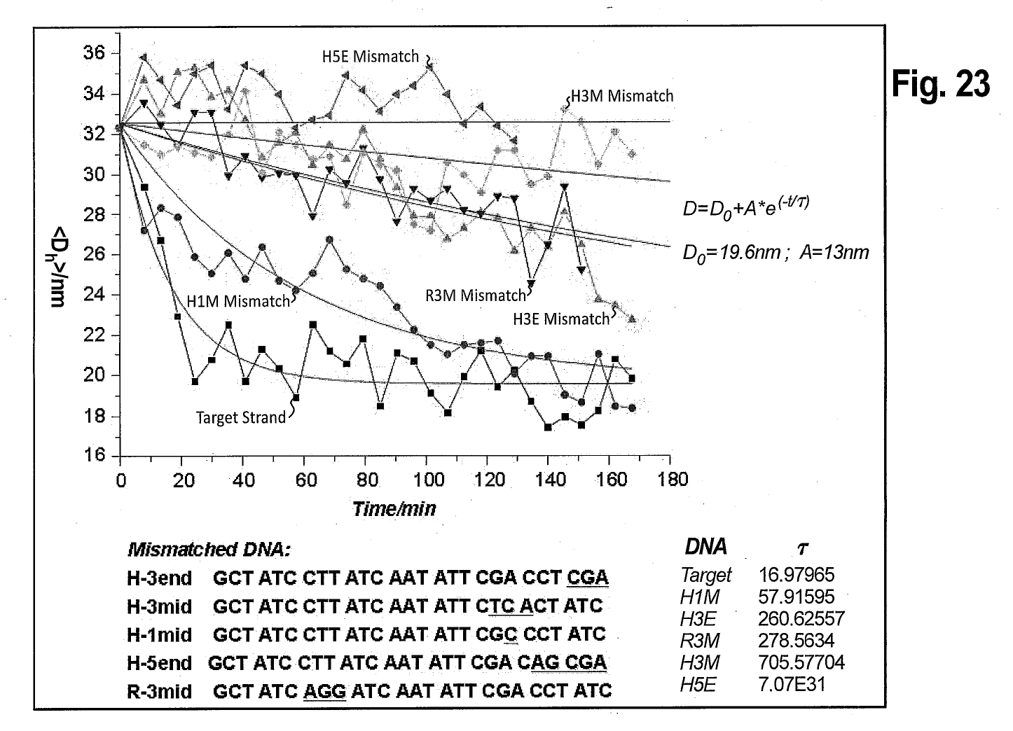

The assembled dimer structures were further probed after assembly and release using dynamic light scattering (DLS). FIG. 12C shows a representative set of DLS profiles characterizing the number averaged hydrodynamic diameter (Dₕ) population for isolated Au (C—Au) and assembled dimers. In contrast to a Dₕ of ≈26 nm for isolated C—Au, the dimer clusters revealed a Dₕ≈37 nm, which is in good agreement with an estimate of 37.4 nm for an ellipsoid formed by two particles separated by 33 bp dsDNA (˜11 nm). Moreover, the size increase of the dimers compared to unassembled monomers can also be observed using gel electrophoresis (FIG. 16), which revealed a decrease in electrophoretic mobility, similar to reported DNA-conjugated nanoparticle groupings.

In addition to the ease in fabrication of nanoparticle dimers, this encoded solid-support based assembly route can also routinely fabricate nanoparticle monomers with strong anisotropic binding character, i.e. Janus particles. The ability to impart nano-objects with controlled anisotropy is currently under a great deal of focus for the fabrication of one-dimensional plasmonic structures and potential for material design. To produce such Janus-monomers, nanoparticles are released from the support after step-2, which allows the linker (A′B′-ssDNA) to impart anisotropy. For instance, large A′/B-capped Au (˜50 nm) were assembled onto the A-Mag via step-1 (FIG. 11A), followed by hybridized with B′C′-linker at a ˜5× ratio. The particle is then released via step-4b using fuel strand as described above. The resulting 5-valent particle thus contains anisotropic character. To probe particle anisotropy and to exploit it for cluster design, these penta valent particles are combined with a ˜5× molar ratio of smaller C—Au, and allowed for assembly overnight. The morphology of the assembly products, as characterized with TEM, is shown in FIG. 12D. The assembled clusters consists of a hemispherical assembly of ˜5 small particles at the large particle, with more than half demonstrating a 5:1 Janus morphology.

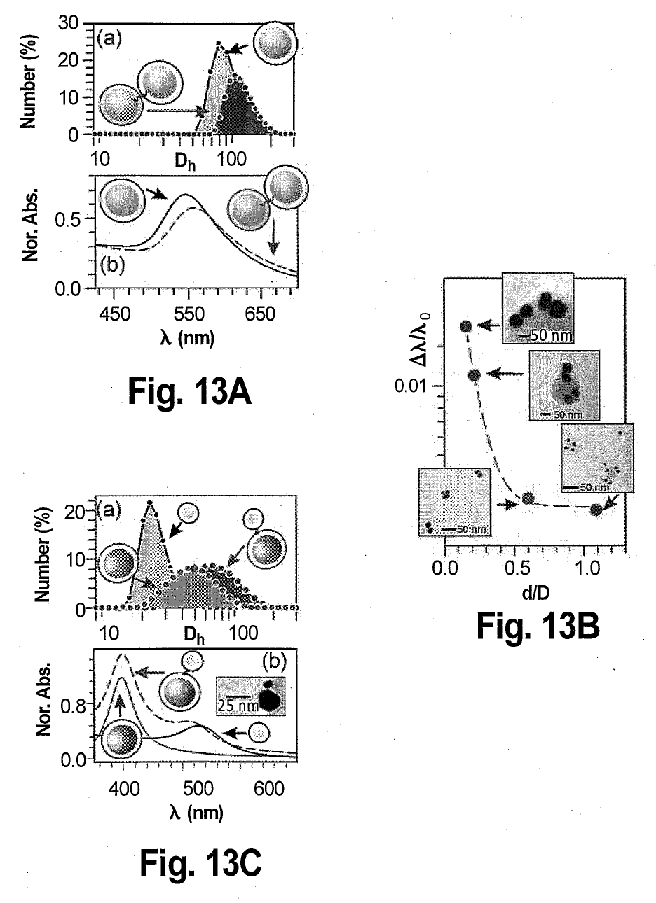

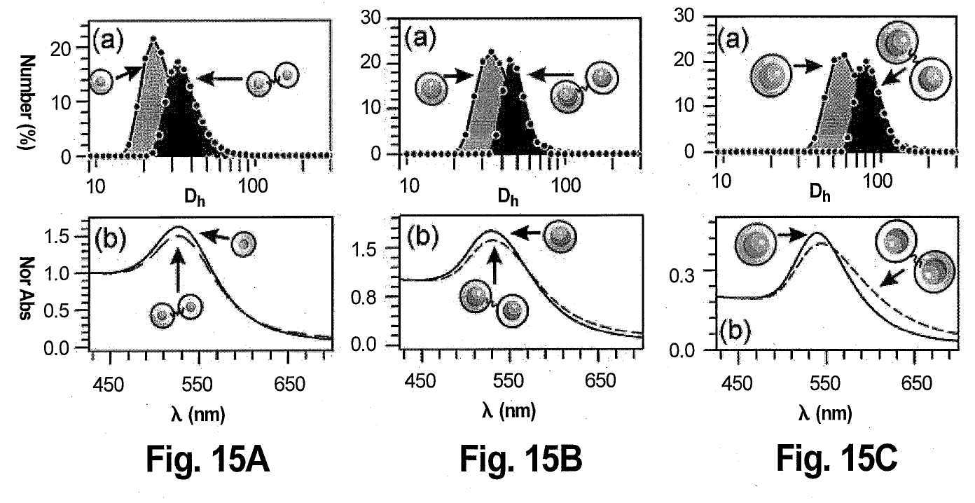

The optical properties of nanoscale clusters, such as dimers, has attracted much interest recently for studies focused on harnessing particle-particle surface plasmon coupling by designing hot spots for surface enhanced raman spectroscopy (SERS), and single molecule detection, and plasmon quenching of fluorescence. The present invention exploits the modularity of its encoded solid-support assembly strategy to produce dimers containing various particle sizes and compositions. Their respective surface plasmon band (SP-band) coupling characteristics via ultraviolet-visible spectrophotometry (UV-vis) were measured. FIG. 13A shows the SP-band for dimers of ˜75 nm Au nanoparticles, fabricated using identical ssDNA and methodology. In contrast to ˜11 nm Au doublets, and ˜20 nm Au doublets (FIG. 15) which showed extinction dampening, but little SP-band shift (1˜2 nm), the 75 nm dimers show increased SP-band coupling, with a red-shift from λ₀=546 nm (isolated Au) to λ˜556 nm. In addition, a slightly lower λ red-shift was observed with ˜50 nm dimers (FIG. 15). This enhanced SP-band coupling is the result of particle diameter (D) becoming larger than interparticle surface-to-surface distance (d). Here, d is held constant, as defined by identical DNA-shells and cross-linker (33-dsDNA, 27-ssDNA), and measured directly via in-situ small angle x-ray scattering (d˜12 nm). FIG. 13B summarizes the relationship between SP-shift (Δλ/λ₀), with interparticle distance to core size (d/D), revealing an exponential dependence. The observed dependence follows the recently described plasmon-ruler equation;

Δλ/λ₀≈C₁exp(−(d/D)/τ)+y₀, where C₁ is a constant, and τ is the decay constant. The observed τ for our dimer systems, 0.14±0.03, is slightly lower than in-plane polarized measurements of gold nanodisks on silicon, fabricated by e-beam lithography.

The encoded solid-support method can also be utilized to produce hetero-clusters of different nanoparticles. FIG. 13C shows the DLS, UV-vis, and TEM results of a Ag—Au dimer, containing 27 nm A′/B—Ag particles 11 nm C—Au. Upon assembly and release, following identical methodology, the assembled dimers possess both Ag SP-band signatures (˜405 nm), and Au SP-bands (˜525 nm). This mixed composition was also confirmed by TEM and DLS (FIG. 13C).

### Materials and Methods

Nanoparticle Synthesis & DNA-functionalization: Gold nanoparticles of 11 nm,(S1) 55 nm, and 75 nm, (S2) were synthesized following literature procedures and quantified using measured extinction coefficients (ε_{11 nm}(λ=518 nm)=1.1×10⁸ cm⁻¹ M⁻¹, ε_{55 nm}=(λ=538 nm) 2.4 10¹⁰ cm⁻¹ M⁻¹, ε_{75 nm}=(λ=545 nm)=4.7×10¹⁰ cm⁻¹ M⁻¹). Thiol-modified single stranded oligonucleotides were purchased from Integrated DNA Technologies Inc. as disulfides, Table S1. Before nanoparticle functionalization, the oligonucleotides were first reduced by dissolving the lyophilized samples (100˜300 nmoles) for 30 minutes with 0.3 ml of a 100 mM dithiothreitol (DTT) solution in purified water or buffer. The reduced DNA was loaded onto a freshly purified sephadex column (G-25, Amersham Bioscience) and eluted with 2.5 ml 10 mM phosphate buffer (pH=7.4). The DNA was quantified using UV-Vis analysis with the specific DNA extinction coefficient. Streptavidin-functionalized magnetic colloids (1-4 μm) were purchased from Pierce Biotechnology Inc., and modified using biotinylated ssDNA following manufacturers directions.

The synthesized Au were functionalized with a ssDNA following methods for high DNA coverage reported by Mirkin and co-workers.(S3) In a typical experiment, an aliquot (1-50 μl) of a purified DNA 50-300 μM solution was added to a 1 ml solution of Au ([Au]=10-30 nM). The ssDNA+Au solutions were incubated at room temperature in a non buffered solution for at least 3 hr before adding phosphate buffer to bring its concentration to 10 mM (pH=7.4). The solution was left to anneal at 25° C. for 4 hr before the addition of NaCl (0.025M). The salt concentration was then increased gradually from 0.025 to 0.3 M NaCl over 24 hr, and left to anneal for an additional 24 hr at 0.3M. The excess DNA next was removed from the solutions by centrifugation for 30 minutes at 4,500 g.

Dimer Fabrication: In a typical experiment, a 0.1M PBS solution (300-1000 μl, 10 mM phosphate buffer, pH=7.4, 0.1M NaCl) consisting of ˜10 nM A′/B—Au is quantified using UV-vis. Next, ˜75 μl of A-Mag ([Mag]˜0.2 mg/ml) is added, and the mixture is allowed to incubate for 3-6 h with stirring. During this process, the gradual decrease in ruby-red color (Au SP-band) to optically transparent, was monitored using UV-vis, demonstrating absorption of A′/B—Au to the solid support. Upon separation via a magnetic field, the supernatant is removed, any un-assembled Au is quantified, and the sample is redispersed with fresh buffer. This separation process is repeated at least 3 times. Next, the B′C′-linker is added at a 3× molar ratio to the assembled A′/B—Au ([B′C′-linker]/[A′/B—Au]=3), and allowed to incubate during mixing for 6-12 h, upon which the system is purified as described above. Next, the C′—Au is added in a strict 1:1 ratio with the assembled concentration of A′/B—Au (i.e., first layer), and allowed to assemble for 6-12 h. The assembly process was again followed with UV-vis. Upon successful second layer assembly, accompanied by a second color change from ruby-red to transparent, beads were separated from a reaction solution. The final assembled product was redispersed in fresh 0.1M PBS, and a 1000× excess of A″-ssDNA fuel strand was added under mixing, and incubated for 3-12 h at room temperature. This process was followed by UV-vis which monitored the release of Au dimers via rise in absorption at 525 nm. Finally, a C″-ssDNA quenching strand was added in a 1:1 ratio of B′C′-ssDNA linker, which passivated any free linkers in the system. The released solution containing dimers were separated from A-Mag via magnetic field, and stored at 4° C. at desired concentrations.

We note that a number of conditions were investigated to optimize this assembly system, and dimer yields. First, we used a low A′/B—Au loading (<10%) on the A-Mag compared to the accessible A-DNA at the A-Mag ([A]˜0.3 nmoles). This increases separation of dimers at the surface, thereby limiting the ability of the second layer of Au (i.e., C—Au) to bridge Au of the first layer, a result which was observed in early experiments at maximum loading. Secondly, a B′C′-ssDNA cross-linker ratio of ˜3× was found to dramatically increased the assembly kinetics and yield of the dimers, compared to a 1× ratio. Third, the use of the C″-ssDNA quenching strand was observed to greatly enhance the stability of the released doublets, limiting additional cross-linking of any free B′C′-ssDNA cross-linkers. Finally, the ease in purification of the system after each assembly step via magnetic separation, greatly enhanced assembly yields, removing excess nanoparticles, DNA cross-links, or impurities from the final dimer solution. In addition, these dimers where also extremely robust, as confirmed by DLS and TEM results for aged samples, which showed little Dₕ change, or decreased morphology yields, respectively.

Janus Particle Fabrication: The assembly and release of Janus-particle monomers was achieved using identical conditions to dimer fabrication. The exception being the particles were disassembled via A″-ssDNA after step-2. The released Janus-particles were quantified using UV-vis, and a desired ratio of C—Au was added, and allowed to assemble free in solution for at least 12 h before sampling for TEM, DLS, and UV-vis.

### Instrumentation:

UV-Visible Spectrophotometry (UV-vis): UV-vis spectra were collected on a Perkin-Elmer Lambda 35 spectrometer (200-1100 nm). Melting analysis was performed in conjunction with a Perkin-Elmer PTP-1 Peltier Temperature Programmer and was performed between 20-75° C. with a temperature ramp of 1° C./min while stirring, in a 10 mM phosphate buffer, 0.21 M NaCl, pH=7.1, buffer solution.

Transmission Electron Microscopy (TEM): TEM micrographs were collected on a JEOL-1200 microscope operated at 120 kV. The samples were prepared by dropcasting an aqueous nanoparticle or dimer solution onto a carbon coated copper grid, followed by the slow removal of excess solution with filter paper after 5 minutes.

Dynamic Light Scattering (DLS): The DLS measurements were obtained with a Malvern Zetasizer ZS instrument. The instrument is equipped with a 633 nm laser source, and a backscattering detector at 173°. ([Au, Dimers]=˜2 nM, 10 mM phosphate buffer, 0.1M NaCl, pH=7.4, T=25° C.).

Dimer Modeling: Diffusion of two connected nanoparticles is approximated as a diffusion of ellipsoidal object with the major axis length (a) of ellipsoid equal to the longest dimension of particles doublet and the minor axis length (b) of ellipsoid equal to size of individual particle. The diffusion coefficient for the ellipsoid D₀ can be described as an average of diffusion coefficients D_{∥} and D⊥ in three orthogonal directions D₀=1/3(D_{∥}+2D⊥) (S4). Direction for D_{∥} is parallel to the major axis of the ellipsoid and directions for D⊥ are perpendicular to the axis.

D_{∥}=kT/f_{∥} and D⊥=kT/f⊥, where f_{∥} and f⊥ are friction coefficients for ellipsoid movements in corresponding directions. The friction coefficients can be related to ellipsoidal dimensions a and b as follows

f_{∥}=16πηa/((a/b)²(2β+a_{∥})) and f⊥=16πηa/(2(a/b)²β+a⊥)

where η is the liquid viscosity, β=cos h⁻¹(a/b)/((a/b)((a/b)²−1)^{0.5}),

a_{∥}=2((a/b)²β−1)/((a/b)²−1), and a⊥=(a/b)²(1−β)/((a/b)²−1)

b was taken as the hydrodynamic size of the single particle (26 nm) and a=2b+l, where l is and estimated separation between monomers in a doublet (˜11 nm). The diffusion coefficient was calculated with the above parameters and the size of equivalent sphere dₑ=kT/(3πηD₀)=37.4 nm was compared with the dimmer size obtained with DLS d=37 nm.

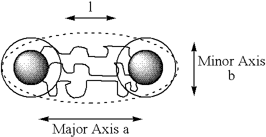

Small Angle X-ray Scattering (SAXS): SAXS experiments were performed in-situ at the National Synchrotron Light Source's (NSLS) X-21A beamline. The scattering data were collected with a MAR CCD area detector and converted to 1D scattering intensity vs. wavevector transfer, q=(4π/λ)sin(θ/2), where λ=1.5498 Å, and θ, are the wavelength of incident X-ray and the scattering angle respectively. The data are presented as the structure factor S(q) vs q. The values of q were calibrated with silver behenate (q=0.1076 A⁻¹) standards. S(q) was calculated as Iₐ(q)/Iₚ(q), where Iₐ(q) and Iₚ(q) are background corrected 1D scattering intensities extracted by angular averaging of CCD images for a system under consideration and un-aggregated Au, respectively. The peak positions in S(q) are determined by fitting a Lorenzian form.

TABLE S1
The ssDNA used in this study.

Sequence (5′ to 3′)

| | |
|---|---|
| A-ssDNA | CTT GTG TCT ACT TCC AAT CCA ATT TTT TTT |
| | TTT TTT TT-Biotin |
| A′-ssDNA | TAC TTC CAA TCC AAT TTT TTT TTT TTT |
| | TTT-C₃H₆-SH |
| B-ssDNA | TTC TCT ACA CTG TCT TTT TTT TTT TTT |
| | TTT-C₃H₆-SH |
| B′C′-ssDNA | AGA CAG TGT AGA GAA AAT ATT GAT AAG GAT |
| | AGC |
| C-ssDNA | HS-C₆H₁₂-TTT TTT TTT TTT GCT ATC CTT |
| | ATC AAT ATT |
| A″-ssDNA | ATT GGA TTG GAA GTA GAC ACA AG |
| C″-ssDNA | ATCCTTATCAATATT |

Thus, it has been demonstrated that the use of bio-recognition between nano-objects at an encoded solid substrate allows for the high-throughput fabrication of both symmetric and asymmetric nanoparticle dimers with regulated interparticle distances, as well as Janus-type nanoparticles. The programmability of DNA motifs, the growing ability to functionalize nanomaterials with DNA, and the modularity of this approach may allow for incorporation of different bio- and nano-topological elements, leading to the development of a technological platform for assembly of rationally designed hetero-architectures.

In summary, a nano-assembly platform using encoded solid supports (NAESS) for the fabrication of high quality assembled nanoparticle doublets, and anisotropic Janus-clusters, using the versatility and tailorability of DNA-binding has been demonstrated. This high throughput approach utilizes a step-by-step assembly at colloidal substrates, which allows for anisotropic growth of doublets or Janus cluster morphologies with high yields under high concentration conditions. The modular nature of this fabrication route make these dimer materials ideal substrates for use in empirically exploring novel surface plasmon resonance phenomenology, changes in biological conformations, dynamically reconfigurable nano systems, as well as in studies related to energy transfer.

This assembly system is simple, allows for high throughput fabrication, and is modular. The method provides a cheap and low maintenance approach to controlling nanoparticle assembly. This system utilizes commercially available ssDNA strands, and is performed in aqueous solution without strenuous environmental controls.

This invention can be used for complex interparticle DNA-scaffolding. In one embodiment, additional classes of nanoparticles, especially semiconductive and magnetic particles, are be employed. In addition, this approach can be utilized for non-DNA systems, including the use of monolayer capped particles, peptides, and hybrid systems.

While the foregoing description has been made with reference to individual embodiments of the invention, it should be understood that those skilled in the art, making use of the teaching herein, may propose various changes and modifications without departing from the invention in its broader aspects.

1. A method of making a nanocluster, the method comprising:
providing a surface comprising at least one anchoring biomolecule, wherein the surface is in a solution;
adding an initial recognition-nano-component to the solution wherein the initial recognition-nano-component comprises i) a nanoparticle and one specifically-bindable-biomolecule, or ii) a nanoparticle and two different types of specifically-bindable-biomolecules, wherein a biomolecule of the initial recognition-nano-component specifically binds to the anchoring biomolecule; and
adding a releasing biomolecule to the solution, wherein the releasing biomolecule binds to the anchoring biomolecule with a greater binding strength than the anchoring biomolecule binds to the initial recognition-nano-component, or wherein the releasing biomolecule binds to the initial recognition-nano-component with a greater binding strength than anchoring biomolecule binds to the initial recognition-nano-component, thereby making a nanocluster.

2. The method of claim 1 further comprising:
(a) providing a plurality of recognition-nano-components, wherein a recognition-nano-component comprises i) a specifically-bindable-nanoparticle, ii) a nanoparticle and one specifically-bindable-biomolecule, or iii) a nanoparticle and two different types of specifically-bindable-biomolecules;
(b) adding a recognition-nano-component to the solution, wherein the recognition nano-component specifically binds to a biomolecule of the initial recognition-nano-component;
(c) subsequently adding a recognition-nano-component to the solution, wherein the recognition nano-component specifically binds to a biomolecule of most recently added recognition-nano-component of the nanocluster; and
(d) repeating step (c) until a desired number of recognition-nano-components are sequentially specifically bonded to the nanocluster.

3. The method of claim 2 further comprising adding a series of capping-moieties to the solution wherein the capping-moieties specifically bind to the unreacted biomolecules of the nanocluster except for the biomolecule of the most recently added recognition-nano-component of the nanocluster.

4. The method of claim 3 further comprising adding an isolating surface to the solution wherein the isolating surface specifically binds to the unreacted biomolecules of the most recently added recognition-nano-component of the nanocluster, and washing away unreacted biomolecules.

5. The method of claim 2 further comprising purifying the solution before a recognition-nano-component is added.

6. The method of claim 5 further comprising adding a series of capping-moieties to the solution wherein the capping-moieties specifically bind to the unreacted biomolecules of the nanocluster.

7. The method of claim 2 wherein the nanocluster comprises about two to about one hundred recognition-nano-components.

8. The method of claim 7 wherein the nanocluster comprises two recognition-nano-components.

9. The method of claim 2 wherein the nanocluster comprises a metal nanoparticle, a semiconductor nanoparticle, an organic nanoparticle, silica, or combinations thereof.

10. The method of claim 9 wherein the metal nanoparticle is a gold nanoparticle, a silver nanoparticle, a copper nanoparticle, a platinum nanoparticle or a palladium nanoparticle.

11. The method of claim 2 further comprising adding a linker to the solution before adding a recognition nano-component, wherein the linker specifically binds to a biomolecule on each of two sequentially added recognition-nano-components, thereby attaching a linker between the two sequentially added recognition-nano-components.

12. The method of claim 11 wherein the linker is added so that the approximate ratio of a linker to a recognition-nano-component is about 1:1 to about 10:1.

13. The method of claim 11 wherein the linker is added so that the approximate ratio of a linker to a recognition-nano-component is about 5:1.

14. The method of claim 2 wherein the specifically-bindable biomolecules of the nanocluster comprise single-stranded nucleic acid molecules; antigens; moieties that bind antigens; or combinations thereof.

15. The method of claim 14 wherein the single-stranded nucleic acid molecules comprises about six to about 200 bases.

16. The method of claim 15 wherein the single-stranded nucleic acid molecules comprises about ten to about thirty bases.

17. A method of detecting the presence of a particular target biomolecule in a sample, the method comprising:
(a) providing a detection dimer, wherein the detection dimer comprises a first recognition nano-component attached to a second recognition nano-component, wherein the first recognition nano-component comprises a first nanoparticle and a first specifically-bindable biomolecule, wherein the second recognition nano-component comprises a second nanoparticle and a second specifically-bindable biomolecule,
(i) wherein the first recognition nano-component is attached to the second recognition nano-component by binding of the first biomolecule to the second biomolecule, wherein the first biomolecule binds to the second biomolecule with an initial binding strength; or
(ii) wherein the first recognition nano-component is attached to the second recognition nano-component by a linker which binds the first biomolecule to the second biomolecule, wherein the linker binds the first biomolecule to the second biomolecule with an initial binding strength;
(b) contacting the detection dimer with the sample, wherein if the target biomolecule is present in the sample,
(i) the target biomolecule binds to either the first biomolecule or the second biomolecule with a detection binding strength, wherein the detection binding strength is greater than the initial binding strength; or
(ii) the target biomolecule binds to the linker with a detection binding strength, wherein the detection binding strength is greater than the initial binding strength;
(c) determining whether the first recognition nano-component became detached from the second recognition nano-component to form monomers, wherein if monomers were formed to a sufficient level, then the target biomolecule is present.

18. A method of detecting the presence of a first target biomolecule and/or a second target biomolecule in a sample, the method comprising:
(a) providing a detection trimer, wherein the detection trimer comprises a first recognition nano-component attached to a second recognition nano-component and a third recognition nano-component,
wherein the first recognition nano-component comprises a first nanoparticle and a first single strand nucleic acid molecule,
wherein the second recognition nano-component comprises a second nanoparticle and a second single strand nucleic acid molecule, and
wherein the third recognition nano-component comprises a third nanoparticle and a third single strand nucleic acid molecule,
wherein the first recognition nano-component is attached to the second recognition nano-component by a single stranded nucleic acid linker which binds i) the portion of the first nucleic acid molecule which is more proximal to the first nanoparticle to ii) the second nucleic acid molecule, wherein the linker binds the first nucleic acid molecule and the second nucleic molecule with an initial binding strength;
wherein the first recognition nano-component is attached to the third recognition nano-component by the binding of i) the portion of the first nucleic acid molecule which is more distal to the first nanoparticle to ii) the third nucleic acid molecule, wherein the first nucleic acid molecule and the third nucleic molecule bind with an initial prime binding strength;
(b) contacting the detection trimer with the sample,
(i) wherein if the first target biomolecule is present in the sample the first target biomolecule binds either the portion of the first nucleic acid molecule which is more proximal to the first nanoparticle or the second nucleic acid molecule or the linker with a detection binding strength, wherein the detection binding strength is greater than the initial binding strength; and
(ii) wherein if the second target biomolecule is present in the sample the second target biomolecule binds either the portion of the first nucleic acid molecule which is more distal to the first nanoparticle or the third nucleic acid molecule with a detection prime binding strength, wherein the detection prime binding strength is greater than the initial prime binding strength; and
(c) determining whether the first recognition nano-component became detached from the second recognition nano-component and/or third recognition nano-component to form dimers and/or monomers, wherein if dimers and/or monomers were formed to a sufficient level, then the first target biomolecule and/or second target biomolecule is present.

19. A method of making a nanoscale architecture, the method comprising:
functionalizing a nanoparticle with single-stranded DNA;
assembling the functionalized nanoparticle onto a DNA-capped magnetic particle, the magnetic particle having dimensions of approximately 100 to 10,000 times those of the nanoparticle;
purifying the assembly of nanoparticles and magnetic particles using a magnetic field;
adding cross-linking strands of single-stranded DNA to the assembly in a ratio of three molecules of cross-linker to one functionalized nanoparticle;
purifying the cross-linker-containing assembly using a magnetic field;
adding a second type of DNA-capped nanoparticle forming an aggregate with the assembly of magnetic particles and nanoparticles;
magnetically separating the aggregates;
liberating species containing two joined nanoparticles; and
removing the magnetic particles.

20. An apparatus comprising:
a joined pair of nanoparticles having anisotropic binding characteristics.
